(19) United States
(12) Patent Application Publication
(10) Pub. No.: US 2008/0213382 A1
(43) Pub. Date: Sep. 4, 2008

(54) THERMOTHERAPY SUSCEPTORS AND METHODS OF USING SAME

(75) Inventors: Robert Ivkov, Ellicott City, MD (US); Allan Foreman, Epping, NH (US)

Correspondence Address: PEPPER HAMILTON LLP, ONE MELLON CENTER, 50TH FLOOR, 500 GRANT STREET, PITTSBURGH, PA, 15219, US

(73) Assignee: TRITON BIOSYSTEMS, INC., Chelmsford, MA (US)

(21) Appl. No.: 12/017,634

(22) Filed: Jan. 22, 2008

Related U.S. Application Data
(60) Provisional application No. 60/885,726, filed on Jan. 19, 2007.

(51) Int. Cl. A61K 9/50; A61P 29/00; A61P 35/00

(52) U.S. Cl. 424/497

(57) ABSTRACT

Untargeted magnetic nanoparticles exhibiting collective behavior and enhanced heating ability in thermotherapeutic applications are described, as are methods for using such untargeted magnetic nanoparticles.

### CROSS-REFERENCE TO RELATED APPLICATIONS

This application claims priority to and benefit of U.S. Provisional Application No. 60/885,726 entitled “Thermotherapy Susceptors, Pharmaceutical Compositions Containing Thermotherapy Susceptors and Methods of Using Same,” filed on Jan. 19, 2007 the entire contents of which is hereby incorporated by reference in its entirety.

### GOVERNMENT INTERESTS

Not applicable

### PARTIES TO A JOINT RESEARCH AGREEMENT

Not applicable

### INCORPORATED BY REFERENCE TO MATERIAL SUBMITTED ON A COMPACT DISC

Not applicable

### BACKGROUND

1. Field of Invention

Not applicable

2. Description of Related Art

Conventional treatments for diseases, such as, for example, cancer and some pathogen based diseases, include treatments that are invasive and may be attended by harmful side effects (e.g., toxicity to healthy cells, disruption of normal bodily function) often making for a traumatic course of therapy with only modest success. For example, conventional treatments for cancer may include surgery followed by radiation and/or chemotherapy. These techniques are not always effective, and even if effective, they suffer from deficiencies such as disfigurement and incomplete removal of effected tissue leading to recurrence. Moreover, radiation therapy and chemotherapy are arduous and are not completely effective against recurrence. Therefore, techniques that are less invasive and traumatic to the patient and are effective only at targeted sites, such as diseased tissue, pathogens or other undesirable matter in the body are desirable.

### BRIEF SUMMARY OF THE INVENTION

Embodiments of the invention described herein are directed to a therapeutic composition including a plurality of untargeted magnetic nanoparticles having an interaction radius of from about 100 nm to about 50 μm and a pharmaceutically acceptable carrier, and in some embodiments the plurality of untargeted magnetic nanoparticles may have an interaction radius of from about 200 nm to about 25 μm.

In various embodiments, the plurality of untargeted magnetic nanoparticles may be stable single-magnetic domain nanoparticles, superparamagnetic particles and combinations thereof, and in such embodiments the untargeted magnetic nanoparticles may be apparently thermally blocked when exposed to a magnetic field and become heated. In some embodiments the plurality of untargeted magnetic nanoparticles may have an average particle, size of less than about 1 μm, and in others the plurality of untargeted magnetic nanoparticles may have an average particle size of from about 0.1 nm to about 800 nm. In certain embodiments, the plurality of untargeted magnetic nanoparticles may have a polydispersity of from about 0.1 to about 1.5. The plurality of untargeted magnetic nanoparticles may be prepared from materials such as, but not limited to, Fe₃O₄, γ-Fe₂O₃, FeCo/SiO₂, Co₃₆C₆₄, Bi₃Fe₅O₁₂, BaFe₁₂O₁₉, NiFe, CoNiFe, Co—Fe₃O₄, FePt—Ag and combinations thereof.

In particular embodiments, the plurality of untargeted magnetic nanoparticles include a core and a coating. In such embodiments the core may include materials such as Fe₃O₄, γ-Fe₂O₃, FeCo/SiO₂, Co₃₆C₆₄, Bi₃Fe₅O₁₂, BaFe₁₂O₁₉, NiFe, CoNiFe, Co—Fe₃O₄, FePt—Ag and combinations thereof, and the coating may include a material such as, but not limited to polymers, biological materials, inorganic coating materials and combinations thereof. In some embodiment, the polymers, may be, for example, acrylates, siloxanes, styrenes, acetates, akylene glycols, alkylenes, alkylene oxides, parylenes, lactic acid, glycolic, acid, hydrogel polymer, histidine-containing polymer, and combinations thereof. In other embodiments, the biological materials may be any of, but not limited to, heparin, heparin sulfate, chondroitin sulfate, chitin, chitosan, cellulose, dextran, alginate, starch, carbohydrate, glycosaminoglycan, extracellular matrix proteins, proteoglycans, glycoproteins, albumin, gelatin and combinations thereof, and in still other embodiments the inorganic coating materials may be metals, metal alloys and ceramics. In certain embodiments the core may be magnetite and the coating may be dextran, and the coating comprises at least two layers of dextran in particular embodiments.

The plurality of untargeted magnetic nanoparticles may have a saturation magnetism of from about 10 kA-m²/g to about 100 kA-m²/g in some embodiments, and in other embodiments the plurality of untargeted magnetic nanoparticles have a specific absorption rate (SAR) of from about 100 W/g to about 1500 W/g when exposed to an alternating magnetic field.

In various embodiments the pharmaceutically acceptable carrier may include, but not be limited to, water, buffered water, saline, Ringer's solution, glycine, hyaluronic acid. dextrose, albumin solution, oils or combinations thereof, and in some embodiments the composition may further include one or more additives, such as, but not limited to, stabilizers, antioxidants, osmolality adjusting agents, buffers, pH adjusting agents, chelants, calcium chelate complexes, salts or combinations thereof. The therapeutic composition of various embodiments may be formulated as a liquid, a gel, an ointment, a lotion, a solid, or a semi-solid. In some embodiments the composition may include targeted magnetic nanoparticles, and in other embodiments the composition may include one or more secondary agents such as, but not limited to, chemotherapeutic agents, radiation therapy agents, vasopermeation enhancement agents, anti-inflammatory agents, anesthetics, analgesics, sedatives, antibiotics and combinations thereof.

Other embodiments of the invention are directed to a method for treating tumorigenic tissue by administering to a patient in need of treatment an effective amount of a therapeutic composition that includes a plurality of untargeted magnetic nanoparticles having an interaction radius of from about 100 nm to about 50 μm and a pharmaceutically acceptable carrier or excipient, and exposing the patient to an energy capable of inducing heating of the plurality of untargeted magnetic nanoparticles. In particular embodiments the plurality of untargeted magnetic nanoparticles may have an interaction radius of from about 200 nm to about 25 μm.

In various embodiments, the tumorigenic tissue may be a solid tumor. Administering in some embodiments may include contacting the tumorigenic tissue with the therapeutic composition directly. In other embodiments, administering may include applying the therapeutic composition directly to the tumorigenic tissue, and in still others administering may include injecting a tumor with the therapeutic composition.

In certain embodiments, such methods may be carried out in combination with radiation therapy, chemotherapy, external beam therapy, surgery, photodymanic therapy (PDT), therapy using biological agents or a combination thereof.

The energy of embodiments may be, for example, as alternating magnetic field (AMF), microwave energy, acoustic energy and combinations thereof, and in particular embodiments the energy may be an alternating magnetic field (AMF). In such embodiments, the alternating magnetic field may have a frequency range of from about 80 kHz to about 800 kHz, and in other embodiments, the alternating magnetic field may have an amplitude of from about 1 kA/m to about 120 kA/m.

Still other embodiments of the invention include a method for treating joint inflammation by administering to a patient in need of treatment an effective amount of a therapeutic composition including a plurality of untargeted magnetic nanoparticles having an interaction radius of from about 100 nm to about 50 μm and a pharmaceutically acceptable carrier or excipient, and exposing the patient to an energy capable of inducing heating of the plurality of untargeted magnetic nanoparticles. In particular embodiments, the plurality of untargeted magnetic nanoparticles may have an interaction radius of from about 200 nm to about 25 μm.

Administering in some embodiments may include contacting inflamed synovial tissue, scar tissue, immune cells and combinations thereof with the therapeutic composition directly. In other embodiments, administering may include applying the therapeutic composition directly to the joint, and in still other embodiments, administering may include injecting the joint with the therapeutic composition. In certain embodiments administering may further include administering one or more of an anti-inflammatory agent, anesthetic, analgesic, sedative, antibiotic or combination thereof.

In various embodiments, the energy may be an alternating magnetic field (AMF), microwave energy, acoustic energy and combinations thereof in some embodiments, exposing may include applying an alternating magnetic field (AMF) to at least a portion of the patient. In certain embodiments, the alternating magnetic field may have a frequency range of from about 80 kHz to about 800 kHz, and in other embodiments the alternating magnetic field may have an amplitude of from about 1 kA/m to about 120 kA/m.

Joint inflammation may vary throughout embodiments and may be caused as a result of, for example, injury, disease, arthritis and combinations thereof. In some embodiments, arthritis may be general arthritis, rheumatoid arthritis, osteoarthritis, tendonitis, bursitis, fibromyalgia and combinations thereof, and in other embodiments disease may include for example, gout, lupus, rickets, ankylosing spondylitis, Sjogrens syndrome and combinations thereof.

### DESCRIPTION OF DRAWING

For a fuller understanding of the nature and advantages of the present invention, reference should he made to the following detailed description taken in connection with the accompanying drawings, in which:

FIG. 1 shows a schematic of an alternating magnetic field solenoid used in in vivo mouse studies.

FIG. 2 shows transmission electron micrographs for Sample A (A.) and Sample B (B.).

FIG. 3 shows SANS/USANS data for Sample A in H₂O (black) and D₂O (dark gray) and Sample B in H₂O (medium gray) and D₂O (light gray). Error bars indicate plus or minus one standard deviation.

FIG. 4 shows the hysteresis loop at 295 K normalized to the mass of iron oxide for Sample A (gray triangles) and Sample B (black circles).

FIG. 5 shows SANS/USANS data for Sample B in 100% H₂O (gray) and 100% D₂O (black). A fit of Sample B in 100% H₂O (light gray line) is also depicted.

FIG. 6 shows SANS data and fits (lines over raw data) for Sample B in 100% H₂O (black), 50% H₂O (light gray), 25% H₂O (dark gray) and 10% H₂O (medium gray).

FIG. 7 shows hysteresis loops at 295 K normalized to the mass of iron oxide for Sample B (black circles) and Sample C (gray triangles). The insert shows a close up of data in a magnetic field at 0 and 86 kA/m (1080 Oe).

### DETAILED DESCRIPTION

Before the present compositions and methods are described, it is to be understood that this invention is not limited to the particular processes, compositions, or methodologies described, as these may vary. It is also to be understood that the terminology used in the description is for the purpose of describing the particular versions or embodiments only, and is not intended to limit the scope of the present invention which will be limited only by the appended claims.

It must be noted that, as used herein, and in the appended claims, the singular forms “a”, “an” and “the” include plural reference unless the context clearly dictates otherwise. Unless defined otherwise, all technical and scientific terms used herein have the same meanings as commonly understood by one of ordinary skill in the art. Although any methods similar or equivalent to those described herein can be used in the practice or testing of embodiments of the present invention, the preferred methods are now described. All publications and references mentioned herein are incorporated by reference. Nothing herein is to be construed as an admission that the invention is not entitled to antedate such disclosure by virtue of prior invention.

As used herein, the term “about” means plus or minus 10% of the numerical value of the number with which it is being used. Therefore, about 50% means in the range of 45%-55%.

“Optional” or “optionally” may be taken to mean that the subsequently described structure, event or circumstance may or may not occur, and that the description includes instances where the event occurs and instances where it does not.

“Administering” when used in conjunction with a therapeutic means to administer a therapeutic directly into or onto a target tissue or to administer a therapeutic to a patient whereby the therapeutic positively impacts the tissue to which it is targeted. “Administering” a composition may be accomplished by injection, infusion, or by either method in combination with other known techniques. Such combination techniques include heating, radiation and ultrasound.

The term “target”, as used herein, refers to the material for which deactivation, rupture, disruption or destruction is desired. For example, diseased cells, pathogens, or infectious material may be considered undesirable material in a diseased subject and may be a target for therapy.

Generally speaking, the term “tissue” refers to any aggregation of similarly specialized cells which are united in the performance of a particular function.

The term “diseased tissue”, as used herein, refers to tissue or cells associated with a diseased state or exhibiting symptoms of a disease including, but not limited to, solid tumor cancers of any type, such as bone, lung, vascular, neuronal, colon, ovarian, breast and prostate cancer. Other types of “diseased tissue” may include tissue of arthritic joints, such as inflamed synovial tissue.

The term “improves” is used to convey that the present invention changes either the appearance, form, characteristics and/or physical attributes of the tissue to which it is being provided, applied or administered.

As used herein, the term “therapeutic” means an agent utilized to treat, combat, ameliorate or prevent an unwanted condition or disease of a patient.

A “therapeutically effective amount” or “effective amount” of a composition as used herein is a predetermined amount calculated to achieve the desired effect.

The term “hyperthermia”, as used herein, refers to heating of tissue to temperatures between about 40° C. and about 60° C.

The term “alternating magnetic field” or “AMF”, as used herein, refers to a magnetic field that changes the direction of its field vector periodically, typically in a sinusoidal, triangular, rectangular or similar shape pattern, with a frequency of in the range of from about 80 kHz to about 800 kHz. The AMF may also be added to a static magnetic field, such that only the AMF component of the resulting magnetic field vector changes direction. It will be appreciated that an alternating magnetic field may be accompanied by an alternating electric field and may be electromagnetic in nature.

The term “energy source”, as used herein, refers to a device that is capable of delivering energy, of a form other than AMF, to a therapeutic for the purpose of activating a potentially radioactive source in the therapeutic.

The tern's “duty cycle”, as used herein, refers to the ratio of the time that an energy source is on to the total time that the energy source is on and off in one on-off cycle.

Thermotherapy may hold promise as a treatment for cancer and other diseases because it induces instantaneous necrosis (typically referred to as “thermo-ablation”) and/or a heat-shock response in cells (classical hyperthermia), leading to cell death via a series of biochemical changes within the cell. Because temperatures from about 40° C. to about 46° C. can cause irreversible damage to diseased cells, and healthy cells are capable of surviving exposure to temperatures up to around 46.5° C., elevating the temperature of cells to between about 40° C. to about 46° C. in diseased tissue may provide a treatment option which selectively destroys diseased cells while not causing damage to normal tissues. Temperatures greater than 46° C. may be effective for the treatment of cancer and other diseases by causing an instantaneous thermo-ablative response.

State-of-the-art systems that employ microwave or radio frequency (RF) hyperthermia, such as annular phased array systems (APAS), attempt to tune energy for regional heating of deep-seated tumors. Such techniques are limited by the heterogeneities of tissue electrical conductivities and that of highly perfused tissue. This leads to the as-yet-unsolved problems of “hot spots” in untargeted tissue with concomitant under-dosage in desired areas. The result is often a lower than expected therapeutic ratio, and an inherent difficulty in determining the heat dose delivered to a desired area with adequate precision. The latter precludes the development of prescriptive clinical protocols which are necessary to ensure reproducible and predictable patient benefits following treatment. All of these factors make selective heating of specific regions with such systems very difficult.

Various embodiments of the invention presented herein are directed to thermotherapeutic compounds, methods for using such thermotherapeutic compounds for treating diseased tissue. The thermotherapeutic compositions described herein may be formulated in any way. For example, in some embodiments, the thermotherapeutic compositions may be formulated as a therapeutic agent that may be delivered to a subject and utilized in a treatment. In other embodiments, the thermotherapeutic compositions of the invention may be formulated as pharmaceutical compositions that may be delivered to a subject as a drug. In still other embodiments, the thermotherapeutic compositions may be administered to a subject in conjunction with the methods for using the thermotherapeutic compounds.

In general, the thermotherapeutic compounds of embodiments include a plurality of magnetic nanoparticles, or “susceptors,” of an energy susceptive material that are capable of generating heat via magnetic hysteresis losses in the presence of an energy source, such as, an alternating magnetic field (AMF). The methods described herein, generally, include the steps of administering an effective amount of a thermotherapeutic compound to a subject in need of therapy and applying energy to the subject. The application of energy may cause inductive heating of the magnetic nanoparticles which in turn heats the tissue to which the thermotherapeutic compounds were administered sufficiently to ablate tissue.

The heat evolved may represent energy loss as the magnetic properties of the material are forced to oscillate in response to the applied alternating magnetic field. The amount of heat generated per cycle of magnetic field and the mechanism responsible for the energy loss depend on the specific characteristics of both the susceptor and the magnetic field. Susceptor heats to a unique temperature, known as the Curie temperature, when subjected to an AMF. The Curie temperature is the temperature of the reversible ferromagnetic to paramagnetic transition of the magnetic material. Below this temperature, the magnetic material heats in an applied AMF. However, above the Curie temperature, the magnetic material becomes paramagnetic and its magnetic domains become unresponsive to the AMF. Thus, the material does not generate heal when exposed to the AMF above the Curie temperature. As the material cools to a temperature below the Curie temperature, it recovers its magnetic properties and resumes heating, as long as the AMF remains present. This cycle may be repeated continuously during exposure to the AMF. Therefore, magnetic materials are able to self-regulate the temperature of heating. The temperature to which susceptor heats may be dependent upon, inter alia, the magnetic properties of the material, characteristics of the magnetic field, and the cooling capacity of the target site.

Many aspects of susceptor, such as material composition, size, and shape, directly affect heating properties, and these characteristics may be designed simultaneously to tailor the heating properties for a particular set of conditions found within a tissue type. For example, the size range and materials of which the susceptors are made may depend upon the particular application. Additionally, selection of the magnetic material and AMF characteristics may be tailored to optimize treatment efficacy of a particular tissue or target type. In various embodiments, susceptors may be prepared that attain a Curie temperature front about 40° C. to about 500° C.

The susceptors of various embodiments, generally, include magnetic nanoparticles or aggregates of magnetic nanoparticles, and in particular embodiments, the susceptors may be single-magnetic domain particles. Any material capable of sustaining a magnetic field may be used to prepare the magnetic nanoparticles of embodiments and any single-magnetic domain particle known in the art may be useful as a susceptor. For example, susceptors may include material such as, but not limited to, magnetite (Fe₃O₄), maghemite (γ-Fe₂O₃) and FeCo/SiO₂, and in some embodiments, susceptors may include aggregates of superparamagnetic grains of, for example, Co₃₆C₆₅, Bi₃Fe₅O₁₂, BaFe₁₂O₁₉, NiFe, CoNiFe, Co—Fe₃O₄, and FePt—Ag, where the state of the aggregate may induce magnetic blocking. In other embodiments, nitrogen-doped Mn clusters such as, for example, MnN or MnₓN_{y}, where x and y are nonzero numbers, may be used as magnetic susceptors. Calculations based on density-functional theory show that the stability and magnetic properties, of small Mn clusters can be fundamentally altered by the presence of nitrogen. Such compositions may be ferromagnetic and have large magnetic moments. Moreover, their binding energies may be substantially enhanced. and the coupling between the magnetic moments at Mn sites may remain ferromagnetic regardless of their size or shape. In still other embodiments, the susceptor may be Nd₁₋ₓCaₓFeO₃. Without wishing to be bound by theory, spontaneous magnetization of the weak ferromagnetism may decrease with increasing Ca content or increasing particle size.

Exemplary susceptors useful in embodiments of the invention include for example, series EMG700 and EMG1111 iron oxide particles of about 110 nm diameter available from Ferrotec Corp. (Nashua, N.H.) which may have a specific absorption rate (SAR) of about 310 Watts per gram of particle at 1,300 Oerstedt flux-density and 150 kHz frequency, and FeCo/SiO₂ particles available from Inframat Corp. (Willington, Conn.) which may have a SAR of about 400 Watts per gram of particle under the same magnetic field conditions.

In some embodiments, the material composition of susceptors may be varied based on the particular target. More specifically, because the self-limiting Curie temperature of a magnetic material is directly related to the material composition, as is the total heat delivered, magnetic particle compositions may be tuned to different tissue or target, types. This may be required because each target type, given its composition and location within the body, possesses unique heating and cooling capacities. For example, a tumor located within a region that is poorly supplied by blood and located within a relatively insulating region may require a lower Curie temperature material than a tumor that is located near a major blood vessel. Targets that are in the bloodstream may require different Curie temperature materials as well. Therefore, susceptors composed of, for example, magnetite may contain other elements such as cobalt, iron, rare earth metals and so on or combinations of additional elements.

In some embodiments, susceptors may be coated to protect the susceptor from the environment of the tissue or to enhance or tune the properties of susceptor. Suitable materials for the coating may include synthetic, biological polymers, copolymers and polymer blends, and inorganic materials.

Examples of polymer materials may include, but not be limited to, various combinations of acrylates, siloxanes, styrenes, acetates, akylene glycols, alkylenes, alkylene oxides, parylenes, lactic acid, glycolic acid, hydrogel polymer, histidine-containing polymer, and combinations thereof. In certain embodiments, the polymer material may be a combination of a hydrogel polymer and a histidine-containing polymer.

Biological materials that may be used to coat susceptors may include polysaccharides, polyaminoacids, proteins, lipids, glycerols, fatty acids and the like, and combinations thereof. For example, biological materials such as heparin, heparin sulfate, chondroitin sulfate, chitin, chitosan, cellulose, dextran, alginate, starch, carbohydrate, glycosaminoglycan and combinations thereof or proteins such as extracellular matrix proteins, proteoglycans, glycoproteins, albumin, gelatin and combinations thereof may be used as coatings for susceptors.

Inorganic coating materials may include, for example, any combination of metals, metal alloys and ceramics such as hydroxyapatite, silicon carbide, carboxylate, sulfonate, phosphate, ferrite, phosphonate, and oxides of Group IV elements of the Periodic Table of Elements. In certain embodiments, these materials may form a composite coating that may contain one or more biological or synthetic polymer. In some embodiments, the coating may also include radioactive or potentially radioactive elements.

In one embodiment, the coating material may be gold. Without wishing to be bound by theory, gold, while being biocompatible, may form a protective coating preventing a chemical change, such as oxidation, in the susceptor. Furthermore, gold may serve as a good conductor enhancing eddy current heating associated with AMF heating. In another embodiment, the gold of a coating may be chemically modified with, for example, a thiol, which may be attached to one or more silane, carboxyl, amine, or hydroxyl group, or a combination thereof. Other chemical methods for modifying the surface of the coating material may also be utilized.

In other embodiments, a coating material, such as those described above, may include one or more transfection agents which may facilitate transport of the susceptor into a cell. For example, in certain embodiments, a coating material may contain vectors, such as plasmids, viruses, phages, or virions, prions, polyaminoacids, cationic liposomes, amphiphiles, and non-liposomal lipids or combination thereof. In other embodiments, the coating material may be a composite or combination of transfection agent with organic and inorganic material and such composites may be tailored for a particular type of a diseased tissue and a specific location within a subject.

In certain embodiments, susceptors may require a protective coating, and the use of a coating material may be important to protect the core material from chemical attack and to protect the subject from toxic effects of the core material. For example, iron, cobalt, other magnetic metals, and their less stable oxides may be coated to prevent oxidation. Moreover, magnetic properties of these minerals may be significantly changed due to oxidation. In a particular example, uncoated magnetite, Fe₃O₄, may undergo oxidation when administered to form maghemite (γ-Fe₂O₃) and eventually hematite (α-Fe₂O₃), and as oxidation occurs, the magnetism of a magnetite susceptor may decrease. In other embodiments, a protective coating may be used where the susceptor material may pose a toxic risk to humans and animals in vivo.

Susceptors, of some embodiments, may additionally include one or more radio active isotope, and the synergistic effects of radiation and heat may be exploited for treating a diseased state. Any radioactive isotope useful for the treatment of disease may be suitable for use in such embodiments and may enhance the therapeutic ratio of the targeted thermotherapy. For example, suitable radioactive isotopes include, but are not limited to, iodine-131, cobalt-60, iridium-192, yttrium-90, strontium-89, samarium-153, rhenium-186, and technetium-99m. The radioactive isotope of some embodiments may be chosen to deliver typical doses of from about 20 Gy to about 60 Gy to the patient. In other embodiments, the radioactive isotope may deliver a sub-lethal dose (less than 20 Gy) prior to thermotherapy and a lethal dose of radiation when thermotherapy is initiated or has been completed. The dose level of radiation may be controlled through choice of radioactive isotope, by controlling the incorporation of a radioactive isotope in the susceptor composition, or a combination thereof. Further controls of the radiation dose may be achieved via the use of a susceptor suspension that includes a mixture of radioactive susceptors and non-radioactive susceptors.

The susceptors of additional embodiments may include one or more isotopes having non-radioactive but unstable nuclei that may possess a high absorption cross-section for subatomic particles, such as, neutrons or protons, and ionizing radiation, for example, x-rays. The nuclei of these isotopes absorb radiation or a subatomic particle causing the nucleus to become unstable and emit radiation as it decays. For example, boron-10 is known to emit radiation upon capturing a neutron. Other isotopes possessing high neutron absorption cross sections include lanthanides, such as, for example, samarium-149, gadolinium-157, and gadolinium-155. In particular embodiments, samarium may be used because it is magnetic, and its incorporation into the magnetic nanoparticle may enhance the magnetic properties of the nanoparticle.

In still other embodiments, susceptors may include one or more imaging isotopes. The magnetic nature of the susceptors described herein may make them suitable contrast agents for magnetic imaging techniques such as Magnetic Resonance Imaging (MRI) or Superconducting Quantum Interference Device (SQUID) based methods. Imaging isotopes may include small paramagnetic or superparamagnetic particles of ferrite, such as, iron oxide Fe₃O₄ or Fe₂O₃. In some embodiments, thermotherapy and an imaging technique, such as, for example, MRI, PET, SPECT, or Bioimpedance may be combined, and visualization may occur prior to, during, or after administration of the susceptors. For example, susceptors may be injected into an organ or tissue of a sample. MRI contrast isotopes may then be used to visualize the target organ or tissue and AMF may be used to destroy the target tissue. In still other embodiments, radiological imaging molecules, such as, but not limited to Molybdenum-99, Technetium-99m, Chromium-51, Copper-64, Dysprosium-165, Ytterbium-169, Indium-111, Iodine-125, Iodine-131, Iridium-192, Iron-59, Phosphorus-32, Potassium-42, Rhodium 186, Rhenium-188, Samarium-153, Selenium-75, Sodium-24, Strontium-89, Xenon-133, Xenon-127, and Yttrium-90, may be incorporated into susceptor. These radiological imaging molecules may then be used to visualize the target organ or tissue and an AMF may be applied to destroy the target tissue or organ.

The susceptor's size may vary among embodiments of the invention. In general, the lower limit for susceptor size may be a diameter below which a single-magnetic domain structure exists. Large magnetic bodies may be divided by domain, or Block walls, into uniformly magnetized regions which minimizes the total energy of the particle, including magnetostatic, exchange, and anisotropic energies, as well as energies contributed by domain walls. The final balance of energies determines both the number and shape of magnetic domains within a magnetic material, and as the size of a magnetic particle is reduced, the size of domains is also reduced. Therefore, domain wall formation also has an associated energy cost that may limit the subdivision of domains to a certain number and size. The lower limit is referred to as a “single-magnetic domain particle” for which the dimensional limit may be in the range of from about 0.1 nm to about 800 nm, depending on the spontaneous magnetization and the anisotropy and exchange energies. However, in some embodiments, the particle size of the susceptor may be up to about 1 μm. In other embodiments the susceptor particle size may be from about 1 nm to about 750 nm, and in still other embodiments, the susceptor particle size may be from about 5 nm to about 500 nm. In certain embodiments the susceptors may have a particle size of from about 10 nm to about 250 nm.

Without wishing to be bound by theory, decreasing grain size may increase the fraction of atoms in a particle that are exposed to the surface of the particle and/or interface regions which may increase the significance of surface and interface electronic structure effects on the magnetic properties of the particle. The intrinsic magnetic properties of a material, such as spontaneous magnetization and magnetocrystalline anisotropy, may, therefore, be strongly influenced by particle size. For example, the total anisotropy energy may increase with decreasing grain size because of a growing surface anisotropy contribution. Additionally, magnetostatic, shape and stress may become increasingly important as the size of the particle is reduced and may combine with magnetocrystalline anisotropy to determine the total anisotropy energy of a single-magnetic domain particle. In various embodiments, enhanced anisotropy may contribute to increased hysteresis losses of these materials when they are subjected to alternating magnetic fields (AMF), and this, in turn, results in higher specific absorption rates (SAR) and improved heating ability.

For example, a magnetic body possessing a single-magnetic domain, i.e., a single-magnetic domain particle, the behavior of the magnetic moment, m, may be governed by the total anisotropy energy of the magnetic grain. The variable m may refer to a vector defining magnitude and direction of magnetization of the magnetic domain with respect to time, environment (temperature, external magnetic field, etc.), and the orientation of the magnetic moment with respect to a crystalline axis of the magnetic nanoparticle. In addition, m may be a product of the anisotropy energy and physical environment, both past and present.

More specifically, the potential of a single-magnetic domain particle to generate heat via hysteresis losses when exposed to an alternating magnetic field (AMF) may be determined by the balance of energies within the particle. Of these, the sum of anisotropy energies presents an energy barrier, E_{B}, to changes in orientation of the magnetic moment, m. Thus, the stability of m with respect to time may increase with increasing values of E_{B}. The grain volume, V, and E_{B} combine to define a characteristic relaxation time, τ₀, an intrinsic property of the particle, which is the time required for spontaneous fluctuations, or relaxations, in the direction of m to some beginning value after it has been forcibly reoriented by a sufficiently strong magnetic field. Therefore, τ₀ may depend on various parameters, such as composition, volume, and shape, of the particle, as well as symmetries within the grain and relaxation pathways available to m.

Anisotropy energy, or potential hysteretic loss, in a single-magnetic domain grain is proportional, in first approximation, to the volume of the grain. Thus, for large single-magnetic domain grains the anisotropy energy may be so high that the energy barrier for magnetization reversal cannot be overcome by thermal energies for any temperature below the material's Curie temperature. A single-magnetic domain particle may be said to be stable when the m of the particle does not fluctuate, and the particle may exhibit an intrinsically stable single-magnetic domain behavior when m does not fluctuate with respect to time. Magnetization reversal in an intrinsically stable single-magnetic domain may occur if the grain is exposed to an external magnetic field that is sufficiently strong to overcome the anisotropy energy forcing a change or reversal of m. Because the anisotropy energy represents a barrier to rotation of the magnetic moment, such a spatial change in this vector is accompanied by a release of energy in the form of heat. The amount of heat released is, therefore, proportional in a first approximation to the anisotropy energy.

Additionally, the amount of heat realized through hysteresis losses of a single-magnetic domain particle when exposed to an AMF may depend on experimental conditions. Experimental temperature will determine the relative difference between E_{B} and energy available to the system, thus setting an experimental relaxation time, or τ. This relationship may be defined by Equation 1:

$$τ = {τ}_{0} exp ( \frac{{E}_{B}}{kT} ) \qquad ( 1 )$$

Where thermal energy is defined by the product kT where k is the Boltzmann constant and T is temperature in Kelvin.

Furthermore, the relationship of the period of oscillation of the AMF, 1/v, where ν is the frequency of oscillation of the AMF, to τ may lead directly to the amount of heat generated through hysteresis losses. For example, when 1/ν is much greater than τ, the magnetic moment appears unblocked and spontaneously overcomes E_{B} and reorients randomly without exhibiting hysteresis losses, and no heat is generated. Conversely, when 1/ν is much less than τ, the magnetic moment appears blocked and resists changes in orientation. Therefore, increasing the magnitude of the frequency of AMF may force m to overcome E_{B} and heat may be released during the change.

When the magnetic field is removed, the magnetic moment will retain the orientation imprinted by the magnetic field for a period of time before reverting to its original orientation. The time required for such an orientation change to occur after the magnetic field is removed may be referred to as the “relaxation time” and is characteristic of the particle as consequence of both the anisotropy energy of the grain and kT. In intrinsically stable single-magnetic domain particles, the relaxation time may be greater than 10⁹ seconds. Hence, the magnetic moment may appear blocked because the anisotropy energy presents an insurmountable barrier to spontaneous rotations of the magnetic spin system for all temperatures up to the material Curie (or Néel) temperature which is defined as the temperature at which a transition from ferromagnetic to paramagnetic state occurs.

As the volume of a particle decreases within the single-magnetic domain, so does the anisotropy energy. Below a certain characteristic grain size, the anisotropy energy may become comparable to, or lower than, kT for any value of T above zero. Because the anisotropy energy is lower than kT at any temperature above zero, it does not present a barrier to magnetization reversal implying that the energy barrier for magnetization reversal may be overcome. The total magnetic moment, of the particle can, therefore, thermally fluctuate about the crystalline axis, similar to a spin in a paramagnetic material allowing a spin system within the single-magnetic domain particles to spontaneously rotate, or spin, while remaining magnetically coupled to the particle. This is commonly referred to as superparamagnetism because of the similarity to paramagnetism observed in bulk materials, and such a single-magnetic domain particle may be said to possess an intrinsically unstable domain, or be intrinsically superparamagnetic.

Exposing a superparamagnetic grain to an external magnetic field will cause the magnetic moment to align in the direction of the magnetic field vector with no concomitant release of energy. When the magnetic field is removed from the grain, the spontaneous fluctuations of the orientation of the magnetic moment will rapidly destroy any imprint imposed by the external magnetic field. The characteristic relaxation time of an intrinsically superparamagnetic grain is, therefore, very short, typically on the order of about 10⁻⁹ seconds, and the magnetic moment of an intrinsically superparamagnetic material is unblocked at all experimental temperatures and for all times longer than the characteristic relaxation time.

Thus, the relaxation time may be defined by temperature, and the magnetic reversal may appear blocked if the measurement time is shorter than the characteristic relaxation time. In this case, the material will exhibit behavior similar to a stable single domain and will generate heat if placed in an AMF with a period that is shorter than the characteristic relaxation time. Such a material may be defined as blocked and apparently stable single domain under these conditions.

Conversely, if the measurement of the AMF period exceeds the characteristic relaxation time of the grain, unblocked or apparently superparamagnetic behavior will be observed. Because the characteristic relaxation time in this instance is much shorter than the time of measurement, or AMF period, magnetization reorientation and reversal occurs randomly with no apparent impedance due to anisotropy energy barriers. Hence, there may be no release of heat.

Temperature is also critically important to distinguishing apparently stable single-magnetic domain, or blocked, behavior from apparently superparamagnetic, or unblocked, behavior. Thus, by analogy, the characteristic relaxation time of the magnetic moment of a particle possessing a single-magnetic domain will appear blocked when exposed to an AMF of fixed period if the experimental temperature, Tₑₓₚ, is below a characteristic value. If Tₑₓₚ is increased to a value above this characteristic temperature, the magnetic moment appears unblocked when exposed to an AMF of the same fixed period. This characteristic temperature may be defined as the blocking temperature, T_{b}. Thus, when a grain possessing a single magnetic domain is placed within an AMF of fixed frequency, the forced oscillations of the magnetic moment may release heat while the grain temperature is below the blocking temperature. Once the grain temperature exceeds the blocking temperature, the magnetic moment becomes unblocked, and any release of heat with further exposure to the AMF may cease. This is because the thermal energy, defined by kT, exceeds the anisotropy energy, thereby providing an excess of energy to the spin system to surmount the magnetocrystalline energy barrier.

The behavior of an individual single-magnetic domain particle is described above. However, embodiments of the invention include magnetic nanoparticles that are a collection of a plurality of susceptors, such as single-magnetic domain particles or a suspension of magnetic nanoparticles suspended in a suitable medium that may have properties that differ from those described above. In such a plurality of susceptors, individual magnetic susceptors may vary in size and may possess one or more than one single-magnetic domain particle that may vary in volume.

A full description of relaxation time and consequent hysteresis losses and heat generated from a suspension of magnetic nanoparticles in suspension in an applied AMF, necessitates inclusion of many more factors than those necessary to describe the behavior of individual single-magnetic domain particles with a fixed volume. Because volume is an intrinsic property of a single-magnetic domain particle that directly affects E_{B}, a determination of τ₀ and τ for a group of particles of varying volumes requires knowledge of the size distribution. While the mean volume may be associated with a value of E_{B} sufficient to block m at a specified temperature and AMF frequency, there may be a sizable fraction of grains in the collection with volume and E_{B} significantly lower. The net effect may result in a measured heat output that is significantly lower than that predicted by the mean volume. Conversely, a collection of particles may possess a mean volume for which the value of E_{B} is lower than that required to block m. This collection may appear superparamagnetic and would not be expected to exhibit hysteresis in an AMF.

Interparticle interaction is another factor that is necessary to fully describe the hysteresis behavior in a collection of single-magnetic domain particles. Magnetic forces are, by definition, long-range forces. That is, the range of influence may extend far beyond the boundary of a magnetic particle. Thus, a collection of more than one single-magnetic domain particle may exhibit properties greater than the sum of the magnetic properties of each individual particle because an additional contribution to the anisotropy energy may result from the collective contribution of the m of each particle with others, and these modified anisotropy energies may produce a collective state that exhibits behavior uncharacteristic of the state of the individual non-interacting particles resulting in an apparently increased E_{B} and a non-homogeneous blocking process. Thus, a collection of magnetic nanoparticles or superparamagnetic particles may appear blocked, and even exhibit hysteresis, under appropriate conditions. However, because the blocking process is non-homogeneous, the observed hysteresis behavior may be considerably weaker than that of a single-magnetic domain particle having a volume comparable to the collection, and the collection cannot be defined as either superparamagnetic or as stable single-magnetic domain because it is neither under all conditions.

Full characterization of a collection of magnetic nanoparticles including stable and superparamagnetic, or purely superparamagnetic single-magnetic domain particles may be difficult and impractical because of the number of measurements required, and the results of some of these measurements may be inconclusive or even contradictory. However, it is possible to define the magnetic properties of a collection of individual magnetic nanoparticles and aggregate magnetic nanoparticles by defining the aggregate mean anisotropy energy. The aggregate mean anisotropy energy may then be used to define the mean characteristic relaxation time and mean behavior of the collection in a specific AMF at a specific temperature. At experimental temperatures between 270 K and 380 K, exposure to an AMF with a frequency in the range of from about 100 kHz to about 600 kHz having amplitude in the range of from about 1 kA/m to about 120 kA/m, and in particular embodiments, from about 7 kA/m to about 105 kA/m, measurement of the SAR may be used to distinguish apparently blocked from apparently unblocked behavior of a collection of individual single-magnetic domain particles and aggregate single-magnetic domain particles. For example, an aggregate of unblocked, or apparently superparamagnetic, particles may, generally, generate less than 10 W/g per particle under the specified conditions. By comparison, an ensemble of non-interacting intrinsically superparamagnetic nanoparticles may generate exactly 0 W/g particles, by definition. Conversely, individual apparently blocked susceptors may generate between 10 W/g to 150 W/g per particle. Further, an aggregate of intrinsically blocked, or stable single-magnetic domain particles may generate greater than 150 W/g particle under the specified conditions via hysteresis heating, even though some superparamagnetic contamination may exist.

The susceptors exhibiting collective behavior may be useful as a platform for heating tissue. For example, untargeted (naked) magnetic nanoparticles such as those described above may be administered directly to a patient in an effective amount at a site of diseased or inflamed tissue. Once administered, the area including the target tissue may be subjected to an AMF and hysteresis heating of the untargeted magnetic nanoparticles may take place. In certain embodiments, the heat generated as a result of the applied AMF may be greater than would be expected from the number and type of particles administered because the collection of particles administered may exhibit collective behavior. Thus, when a collection of susceptors are administered to a target tissue, the heat resulting from the application of an AMF to the target tissue may be enhanced.

As used herein, the terms “untargeted” or “naked” susceptors refers to susceptors that have not been modified to interact with a specific cell type or molecule, in contrast, a “targeted” susceptor may be modified to interact with a specific molecule using, for example, an antibody that is covalently attached to the susceptor. Untargeted or naked susceptors contain no such targeting mechanism.

In various embodiments, the magnetic susceptors administered may exhibit collective behavior or be present in the tissue in a collective magnetic state. Individual magnetic susceptors move through solutions as loose aggregates in which the particles are in close proximity to one another but not physically contacting one another. Without wishing to be bound by theory, untargeted magnetic susceptors having particular properties may achieve a collective magnetic state in biological tissues. Therefore, when administered, these susceptors may form collections in the cells of the tissue or in the space between cells. Moreover, without wishing to be bound by theory, the concentration of magnetic nanoparticles may have a direct effect on the total heat produced.

Magnetic susceptors capable of achieving a collective magnetic state may vary in size, shape or polydispersity and may possess a variety of magnetic properties. For example, in one embodiment, magnetic susceptors capable of achieving a collective magnetic state may have an interaction radius of less than 75 mm. In another embodiment, the magnetic susceptors may have an interaction radius of from about 100 nm to about 50 μm, and in still another embodiment, the magnetic susceptors may have an interaction radius of from about 200 nm to about 25 μm. Such particles may act as loose aggregates or clusters of individual particles that do not physically interact.

The magnitude of the interaction radius of various magnetic susceptors embodied by the invention may be altered by various methods known in the art. In some embodiments, the interaction radius may be decreased by proving at least two or more layers of a coating material to the core of a magnetic susceptor. For example, in one embodiment, two layers of dextran may be applied to a magnetite susceptor to reduce the interaction radius such that it is within an acceptable range to allow for a collection of such particles to achieve a collective magnetic state. In other embodiments, any of the polymeric or biological coating materials described above may induce a similar effect on the interaction radius.

In still other embodiments, coated particles may form a network of coating material that allows the magnetic nanoparticles to exhibit collective behavior. For example, in one embodiment, dextran coated magnetic nanoparticles may form a dextran network even though the dextran coating would normally cause the coated particles to repel one another. Without wishing to be bound by theory, the magnetism exhibit by the coated particles may allow the dextran coating network to form which stimulates collective behavior in the magnetic nanoparticle cores,

Magnetic susceptors achieving a collective magnetic state may also exhibit a specific absorption rate (SAR) when a magnetic field is applied to the susceptors that is greater than the SAR of the combined individual susceptors or an aggregate in which the particles physically interact. For example, in some embodiments, a plurality of magnetic susceptors acting in a collective magnetic state may achieve an SAR of from greater than 150 W/g to about 1750 W/g or, in others, from about 175 W/g to about 1500 W/g based on the iron content. In still other embodiments, the SAR may be greater than 1500 W/g and may depend on the material used to prepare the magnetic susceptors. Moreover, the increase in SAR noted above may be adjusted based on the number of particles in the collection. For example, in embodiments in which a large collection of untargeted magnetic susceptors are administered the SAR may be amplified when compared to embodiments in which a smaller collection of untargeted magnetic susceptors are administered. Thus, the increased heating ability of magnetic susceptors may be concentration dependent.

Without wishing to be bound by theory, the saturation magnetism of the particles may not contribute to the enhanced heating ability of magnetic susceptors exhibiting collective magnetic behavior, and in various embodiments the saturation magnetism may be from about 10 kA-m²/g to about 100 kA-m²/g. The size of the magnetic susceptors may also not have a direct effect on the collective magnetic behavior, and therefore, particles utilized in embodiments of the invention may have a particle size of less than about 0.1 μm as described above. It is additionally noted that particle size may directly affect healing as described above. Additionally, susceptors that act in a collective magnetic state may have a broad polydispersity without affecting the collective behavior. For example, in one embodiment, the polydispersity of a collection of susceptors may be from about 0.1 to about 1.5.

In particular embodiments, a plurality of untargeted magnetic susceptors exhibiting a collective magnetic state may include one or more targeted magnetic susceptors which maintain a collective magnetic state with the untargeted susceptors when in solution. Without wishing to be bound by theory, such combined untargeted and targeted susceptor collections may allow for the collection of be targeted to specific tissue, cell or protein without compromising the collective magnetic state. Thus, the advantages exhibited by untargeted susceptors may be passed to targeted systems.

Untargeted susceptors may be used to treat any number of disease indications for which heating may provide a form of treatment, and embodiments of the invention include methods for treating a number of maladies. In various embodiments, untargeted susceptors may be administered directly to diseased tissue to treat diseased states in which heat may be applied to a tissue to ablate tissue. For example, in one embodiment, untargeted susceptors may be administered directly to a solid tumor by, for example, direct injection, and an AMF may be applied to the portion of the patient containing the tumor. The susceptors may exhibit a collective magnetic state in the tumor resulting in heating and ablation of the tumor tissue thereby reducing or eliminating the tumor tissue. Treatment using susceptors exhibiting a collective magnetic state may be used to treat any type of cancer, and in certain embodiments such treatment is used to treat localized solid tumors, such as, for example, cancers of the skin, head and neck, tongue, throat, larynx, brain, breast, liver, pancreas, lymph nodes, joint or synovial, uterine or cervix, peritoneum or other specific organ cancers and the like. In other embodiments, susceptors exhibiting a collective magnetic state may be used to treat leukemia and lymphoma (cancers of the blood-forming cells and lymphatic system, respectively).

In another exemplary embodiment, untargeted susceptors may be administered to treat joint inflammation and/or joint swelling by, for example, direct injection into the synovial tissue of the joint. The susceptors may exhibit a collective magnetic state and become heated when an AMF is applied. The heated susceptors may ablate scar tissue or inflamed synovial tissue in the joint, thereby reducing or eliminating the symptoms. Susceptors exhibiting a collective magnetic state may be useful for treating any type of joint inflammation including, for example, arthritis, and any form of arthritis, known may be treated in such a way including, but not limited to, general arthritis, rheumatoid arthritis, osteoarthritis, tendonitis, bursitis and fibromyalgia. In other embodiments, such susceptors may be used to treat other inflammatory diseases, such as, for example, swelling, gout, lupus, rickets, ankylosing spondylitis, Sjogrens syndrome and the like. In still other embodiments, such susceptors may be used to treat injury.

In still other embodiments, the untargeted susceptors may be applied directly to diseased tissue or an area surrounding the diseased tissue using a gel, lotion, ointment, salve or wash. For example, in one embodiment, following a surgical procedure to remove diseased tissue such as, for example, a tumor, the area surrounding the tumor may be washed in an ointment, gel, lavage or solution including the untargeted susceptors. An AMF may then be applied to the area and tumorigenic tissue remaining at the site following the tumorectomy may be ablated or destroyed. Similarly, a gel, ointment or solution including untargeted susceptors may be used to reduce or eliminate infection or inflammation at an incision site or any surgical procedure. In yet other embodiments, a gel, ointment, lotion or salve, may be applied directly to the skin of a patient, and an AMF may be used to apply heat to the tissue of the patient without ablating tissue, for example, to treat joint or muscle pain or stiffness.

The untargeted susceptors of various embodiments described above may be mixed into a solution appropriated for administration into a patient such as, for example, water or saline or prepared as a therapeutic formulation including other components or active agents. Untargeted susceptors of the invention may be formulated as a therapeutic composition according to techniques known and practiced in the art. “Therapeutic compositions” are generally characterized as being at least sterile and pyrogen-free, and as used herein, the terms “therapeutic formulations” or “therapeutic composition” include formulations for human and veterinary use. Therapeutic compositions of various embodiments of the invention may be prepared as described in Remington's Pharmaceutical Science, 17th ed., Mack Publishing Company, Easton, Pa. (1985), the entire disclosure of which is herein incorporated by reference.

The therapeutic compositions encompassed by embodiments of the invention may vary. For example, in some embodiments, therapeutic formulations of the invention may include from about 0.01% to about 95% by weight of untargeted susceptors mixed with a physiologically acceptable carrier medium such as, for example, water, buffered water, normal saline, 0.4% saline, 0.3% glycine, hyaluronic acid and the like. In other embodiments, the untargeted magnetic susceptors may make up from about 1% to about 90% by weight of a therapeutic composition. Other embodiments of therapeutic compositions may include stabilizers such as appropriate pharmaceutical grade surfactants, e.g. TWEEN, and saccharides, e.g. dextrose, may also be incorporated into such therapeutic compositions. Therapeutic compositions encompassed by the invention may also include conventional pharmaceutical excipients and/or additives. For example, suitable pharmaceutical excipients may include stabilizers, antioxidants, osmolality adjusting agents, buffers, and pH adjusting agents, and suitable additives may include physiologically biocompatible buffers (e.g., tromethamine hydrochloride), additions of chelants (such as, for example, DTPA or DTPA-bisamide) or calcium chelate complexes (as for example calcium DTPA, CaNaDTPA-bisamide), or optionally, additions of calcium or sodium salts (for example, calcium chloride, calcium ascorbate, calcium gluconate or calcium lactate). Such therapeutic compositions of the invention may be packaged for use in liquid or gel form, and in certain embodiments, such therapeutic compositions may be lyophilized. In particular embodiments, untargeted susceptors may be prepared in an injectable form (suspension, emulsion) in a medium such as, for example, water, saline, Ringer's solution, dextrose, albumin solution or oils.

In other embodiments, therapeutic compositions of the invention may be packaged for use as a solid, semisolid, suspension, dispersion, or emulsion. Conventional nontoxic solid earners may be incorporated into such compositions and may include, for example, pharmaceutical grades of mannitol, lactose, starch, magnesium stearate, sodium saccharin, talcum, cellulose, glucose, sucrose, magnesium carbonate and the like. For example, about 1 to about 95% by volume or, in a further example, 25% to about 75% by volume of any of the carriers and excipients listed above may be mixed with the untargeted susceptors of the invention.

In some embodiments, the therapeutic compositions of embodiments may additionally include one or more secondary active agents. For example, in one embodiment, one or more chemotherapeutic agents may be combined with the untargeted susceptor to enhance a therapeutic efficiency of the susceptors. Examples of chemotherapeutic agents suitable for such uses may include, but are not limited to, alkylating agents, plant alkaloids, anti-tumor antibiotics, antimetabolites, topoisomerase inhibitors, hormonal agents, growth factors, cytokines, mitotic inhibitors and combinations of these. In particular embodiments, the chemotherapeutic agent may be one or more of carmustine (BCNU), 5-fluorouracil (5-FU), cytarabine (Ara-C), gemcitabine, methotrexate, daunorubicin, doxorubicin, dexamethasone, topotecan, etoposide, paclitaxel, vincristine, tamoxifen, thalidomide, melphalan, cyclophosphamide, alkyl sulfonates, nitrosoureas, ethylenimines, triazenes, folate antagonists, purine analogs, pyrimidine analogs, anthracyclines, bleomycins, mitomycins, dactinomycins, plicamycin, vinca alkaloids, epipodophyllotoxins, taxanes, glucocorticoids, L-asparaginase, estrogens, androgens, progestins, luteinizing hormones, octreotide actetate, hydroxyurea, procarbazine, mitotane, hexamethylmelamine, carboplatin, mitoxantrone, monoclonal antibodies, levamisole, interferons, interleukins, filgrastim, sargramostim, and platinum complexes such as cisplatin carboplatin and oxaliplatin and the like. Further examples of chemotherapeutic agents are described in “Modern Pharmacology with Clinical Applications”, Sixth Edition, Craig & Stitzel, Chpt. 56, pg 639-656 (2004), herein incorporated by reference in its entirety. In another embodiment, the therapeutic compositions of embodiments may additionally include one or more anti-inflammatory agent, one or more anesthetic, one or more analgesic, one or more sedative, one or more antibiotic and the like or a combination thereof.

The untargeted susceptors or therapeutic compositions containing untargeted susceptors of embodiments described herein may be administered by any method known in the art, and dosage may depend upon, for example, the type and location of the diseased tissue. For example, untargeted susceptors may be administered parenterally by methods including, but not limited to, intravascular administration, peri- and intra-tissue injection, subcutaneous injection or deposition, or subcutaneous infusion, intraperitoneal injection, intraorgan injection, intramuscular injection and direct administration at or near a site of diseased tissue to facilitate efficient treatment of the diseased tissue. In particular embodiments, intravascular administration may include intravenous bolus injection, intravenous infusion, intra-arterial bolus injection, intra-arterial infusion and catheter instillation into a vasculature, and in other embodiments, peri- and intra-tissue injection may include intra-tumor injection and intra-synovial or intra-joint injection. For example, subcutaneous injection may be used to deliver susceptors to a tumor in breast tissue, or intraorgan injection may be used to deliver susceptors to a tumor in liver tissue. In other embodiments, susceptors may be delivered by a wash, lavage, as a rinse with sponge, or other surgical cloth as a perisurgical administration technique. For example, a sponge having absorbed susceptors in a suspension, salve or lotion may be used to swab tissue following removal of a cancerous growth.

Untargeted susceptors of the invention can be administered in a single dose or in multiple doses in various embodiments of the invention. One skilled in the art can readily determine an appropriate dosage regimen for administering susceptors to a given subject, and the dosage regimen may vary depending upon the diseased state and the health of the individual. For example, the untargeted susceptors can be administered to the subject once as, for instance, a single injection or deposition at or near the site of diseased tissue. Alternatively, untargeted susceptors can be administered once or twice daily to a subject for a period of from about one to about twenty-eight days, or from about one to about ten days. For example, in some embodiments, untargeted susceptors may be injected at or near the site of diseased tissue once a day for up to seven days. Once administered to the patient, delivery of susceptor to a target site may be assisted by applying a static magnetic field to the target area due to the magnetic nature of the susceptors. Assisted delivery may depend on the location of the target.

The energy may be applied to a targeted cell, targeted tissue, either intracorporeally (inside the body) or extracorporeally (outside the body) or energy may be applied to a portion of the subject's body or the entire body. Application of the energy may commence immediately upon completion of a single administration of the susceptors, and may be repeated daily after each administration or after the completion of several administrations. Alternatively, induced heating may begin after a period of time, for example, several minutes to several days after completion of administration of the susceptors. Duration of each induced healing session may be from five minutes to five hours. Without wishing to be bound by theory, the period of time from administration of the susceptors to energy application may allow the susceptors to be taken up by cells within the target tissue. For example, in some embodiments target tissue may include cells involved in inflammation such as, for example, monocytes or leukocytes, which may engulf the particles after administration. Applying energy to cells that have engulfed susceptors may increase the likelihood of these cells being destroyed as a result of heating and, thus, reduce inflammation.

A variety of forms of energy may be applied to the patient by any means known in the art to provide induced heating of the untargeted susceptors including, but not limited to, AMF, microwave energy, acoustic energy or a combination thereof. For example, in one embodiment, AMF at the appropriate frequency and amplitude may be applied to a patient, and in another embodiment, microwave energy at the appropriate frequency may be applied. In still another embodiment an additional energy may be used in combination with AMF, microwave or acoustic energy which may allow a susceptor to discharge ionising radiation (e.g., neutron, alpha, beta, gamma, etc.). In particular embodiments, AMF energy may be applied to a subject to induce heating of untargeted susceptors to produce therapeutic heating of the untargeted susceptors, and in such embodiments, the frequency of the AMF may be in the range of about 80 kHz to about 800 kHz.

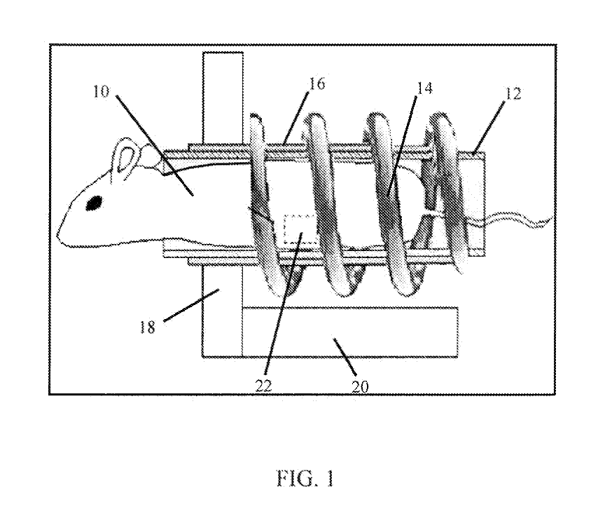

Various sources for AMF, microwave and acoustic energy are available in the art, and any such source may be utilized in embodiments of the invention. For example, in some embodiments, the devices described in U.S. applications Ser. Nos. 10/176,950 and 10/200,082, hereby incorporated by reference in their entireties, may be used as a source of AMF energy which may be broadly applied to a subject to induce heating of the susceptors of the invention. In other embodiments, sources used to generate the AMF may provide a focused and/or a homogeneous field. For example, in one embodiment, a magnetic solenoid coil as depicted in FIG. 1 may be used for heating susceptors which have been administered to tissue in a portion of a subject, such as human limbs or small animals. For example in FIG. 1, a mouse 10 to which susceptors have been administered locally to a particular tissue 22 is retained in a tube 12 that is wrapped in a magnetic solenoid coil 14. A felt liner 16 surrounds the tube 12 and acts to pad the tube 12. A flux concentrator ring 18 surrounds a portion of the tube 12 and is connected to a flux concentrator base 20. The magnetic coil 14 in such embodiments may be a coil, as depicted in FIG. 1, or circular, doughnut shaped ring of low reluctance magnetic material, which may be specifically formulated for magnetic cores operating at a desired frequency. For example, an operative frequency in some embodiments may be from about 80 kHz to about 800 kHz or, in certain embodiments, at about 150 kHz. This approach allows for higher magnetic field strength for application to the subject and reduced eddy current heating. In addition, a circular doughnut shaped ring and a focusing bar may cause the field strength of the magnetic field to drop off significantly outside of solenoid coil. Therefore, a magnetic solenoid coil may focus the AMF while protecting the non-targeted parts of the subject, such as the head and vital organs.

In other embodiments, microwave resonance heating may be used to heat susceptors administered to a subject through resonance heating. In such embodiments, a susceptor material may be selected such that the internal chemical bonds of the material may resonate at a particular frequency or by exploiting interactions of the microwave energy with materials that possess particular magnetic, electrical or electric dipole structures. In general, resonance heating may be advantageous because the targeted material absorbs large quantities of energy from a relatively low power energy source. Thus, non-targeted materials such as tissue may have a resonance frequency that differs from that of the susceptors and may not heat to the same extent. In addition to direct modes of heating, resonance heating may be used indirectly. For example, in one embodiment, susceptors may be selected that possess magnetic or electric properties that may induce a shift in the resonance frequency of the tissue to which they become attached. Thus, the molecules of the tissue in close proximity to the susceptors will preferentially heat when an energy field tuned to the appropriate frequency is applied to the tissue.

In certain embodiments, disease tissue removed from the subject and energy may be applied to the tissue extracorporeally. In such embodiments, the untargeted susceptors may be administered to the subject prior to removal of the diseased tissue or untargeted susceptors may be applied to the diseased tissue following removal. As in previously described embodiment, exposing the diseased tissue including untargeted susceptors to an energy source may cause portions of the diseased tissue to lyse, denature, or otherwise become damaged thereby treating the diseased tissue. The treated tissue may then be returned to the body of the subject. For example, in one embodiment, the extracted tissue may be blood, and susceptor containing target cells carried in blood serum or blood plasma may be separated extracorporeally from the other blood components and exposed to an energy source to destroy or inactivate the target. Following exposure, the treated components may be recombined with the other blood components and returned to the subject's body. In another embodiment, the susceptors may be introduced into extracted tissue while the extracted tissue is outside of the subject's body or body part. For example, extracted blood from the subject may be introduced to susceptors in blood circulating outside of the body prior to exposure to an energy source. In yet another embodiment, susceptors may be contained in a vessel or column through which the blood, blood serum or blood plasma flows. The vessel or column may be exposed to an energy source so as to destroy or inactivate the targeted cells prior to returning the blood to the subject's body.

The advantages of providing energy to the susceptors extracorporeally may include the ability to heat to higher temperatures and/or heat more rapidly to enhance efficacy while minimizing heating and damage to surrounding body tissue, and the ability to reduce exposure of the body to the energy from the energy source. In embodiments where the susceptors are introduced into the blood circulating outside of a subject's body, the blood serum or blood plasma that is extracted from the body, susceptors need not be directly introduced into the body, and higher concentrations of susceptors can be introduced to target. Further, the portion of the subject that is being treated extracorporeally can be cooled externally, using a number of applicable methods, while energy may be provided to the susceptors without mitigating the therapeutic effect. In addition, the cooling may take place before, and/or after the administration of energy.

In still other embodiments, treated susceptors and the associated targets need not be returned to the subject's body. For example, if the susceptors and the associated targets are contained in blood extracted from a subject, the treated susceptors and the associated targets may be separated from the blood prior to returning the blood to the subject's body. In embodiments where the susceptors contain a magnetic component, the tissue containing susceptors may be passed through a magnetic field gradient to separate susceptors and the associated tissue from the extracted tissue. In doing so, the amount of susceptors and treated disease material returned to the subject's body is reduced.

In yet another embodiment of extracorporeal treatment, the tissue selected for heating is completely or partially removed from a subject's body during a surgical procedure. The tissue can remain connected to the body or can be dissected and reattached after the therapy. In still another embodiment, the tissue is removed from the body or body part of one donor subject and transplanted to that of a recipient subject after the therapy.

The various embodiments of susceptors and methods of treating diseased tissue described herein above may be used alone, or in combination with another form of therapy. For example, susceptors may be introduced into diseased tissue prior to, during, or after treatments including, but not limited to, radiotherapy, chemotherapy, external beam therapy, surgery, photodymanic therapy (PDT), therapy using biologics or any combination of therapies.

In one embodiment of the present invention, radiotherapy or radiation therapy may be used in combination with thermotherapy methods disclosed herein. Radiotherapy may be applied at least once prior to, during, or after susceptor administration, or any combination thereof. Radiotherapy, also referred to as radiation therapy, is the treatment of cancer and other diseases utilizing ionizing radiation. Ionizing radiation deposits energy that injures or destroys cells in the area being treated (the “target tissue”) by damaging their genetic material, making it impossible for these cells to continue to grow. Although radiation damages both cancer cells and normal cells, uninfected cells may be able to repair themselves and function properly.

In one embodiment, x-ray or gamma ray therapy may be utilized. Depending on the amount of energy they possess, x- or gamma rays can be used to destroy cancer cells on the surface of or deeper in the body with higher energy x- or gamma ray beams being used for deeper penetration into the target tissue.

In one embodiment of the invention, external beam radiotherapy is used in combination with the thermotherapy methods disclosed herein. In such embodiments, machines to focus radiation such as x-rays may be used on a cancer for a type of therapy commonly referred to as external beam radiotherapy. The beams may be shielded from the outside world and special shielding is used for “focusing” these beams onto defined body areas. In some embodiments, thermotherapy and radiotherapy methods may be used simultaneously, and an AMF system may include a separate opening for an x-ray beam to enter. Alternatively, the beam may be directed through an opening in the patient (patient gantry). In another embodiment, a large dose of external radiation may be directed at the susceptor treated tumor and surrounding tissue during surgery, for an intraoperative irradiation technique.

Gamma rays may be utilized in any embodiment described above in place of x-rays. Gamma rays are produced spontaneously as certain elements (such as radium, uranium, and cobalt 60) release radiation as they decompose or decay. Each element decays at a specific rate and emits energy in the form of gamma rays and other particles. X-rays and gamma rays generally have the same effect on cancer cells.

Another embodiment includes the use of particle beam radiation therapy in combination with thermotherapy, and in one embodiment, high LET therapy is used in combination with the targeted thermotherapy methods disclosed herein. During particle beam therapy, fast-moving subatomic particles generated by particle accelerators may be used to treat localized cancers. Some particles (neutrons, pions, and heavy ions) deposit more energy than x-rays or gamma rays along the path they take through tissue, thus causing more damage to the cells they contact. This type of radiation is often referred to as high linear energy transfer (high LET) radiation.

In still other embodiments, radiation may be delivered to cancer cells through radioactive implants placed directly in or on a tumor or in a body cavity, and in another embodiment of the invention, internal radiotherapy is used in combination with the targeted thermotherapy methods disclosed herein. This is referred to as internal radiotherapy, and is commonly used for, for example, brachytherapy, interstitial irradiation, and intracavitary irradiation types of internal radiotherapy. During this treatment, the radiation dose is concentrated in a small area. In such embodiments, the implant may include a material that heats during the AMF treatment by eddy current or hysteretic heating, or that does not heat under AMF exposure, such as plastic, ceramic, glass, or transplanted human tissue.

In still another embodiment, radiolabled antibodies, which when injected into a subject actively seek out the cancerous cells and destroy the cells using radiation, may be used to deliver doses of radiation directly to the cancer site in combination with targeted thermotherapy. In some such embodiments, the radiolabeled antibody may be administered separately from susceptors, and in others, the radio-labelled antibody may be administered simultaneously with susceptors. In still others, at least one radioisotope may be attached to a susceptor, and the susceptor can be a dual therapy susceptor.

Examples of radioisotopes suitable for use herein include, but are not limited to, molybdenum-99, technetium-99m, chromium-51, cobalt-60, copper-64d, dysprosium-165, ytterbium-169, iodine-125, iodine-131, iridium-192, iron-59, phosphorus-32, potassium-42, rhenium-188 (derived from Tungsten-188, samarium-153, selenium-75, sodium-24, strontium-89, xenon-133, xenon-127, yttrium-90

In some embodiments, thermotherapy as described above may be used in combination with chemotherapy. Chemotherapy is the treatment of diseases, such as cancer, with drugs. For most types of cancer, chemotherapy often requires the use of a number of different drugs or agents; this is referred to as combination chemotherapy. In various embodiments, chemotherapy may be administered in any way known in the art, such as, for example, intravenously (IV: into a vein is the most common), intramuscularly (IM; injection into a muscle), orally (by mouth), subcutaneously (SC; injection under the skin), intralesionally (IL; directly into a cancerous area), intrathecally (IT; into the fluid around the spine), topically (application onto the skin) and the like. Tumor cell resistance to various chemotherapeutic agents represents a major problem in clinical oncology.

Chemotherapeutic agents that may be used in embodiments of the invention may stop the cell cycle at any stage, for example, during S, M, G₁ or G₂. For example, S phase-dependent agents may include, antimetabolics, such as, Apercitabine, Cytarabine, Doxorubicin, Fludarabine, Floxuridine, Fluorouracil, Gemcitabine, Hydroxyurea, Mercaptopurine, Methotrexate, Prednisone, Procarbazine, and Thioguanine. M phase-dependent agents include vinca alkaloids, such as, Vinblastine, Vincristine, and Vinorelbine; podophyllotoxins, such as, Etoposide, and Teniposide; taxanes including Doxetaxel; and Paxlitaxel. G₂ phase-dependent agents include Bleomycin, Irinotecan, Mitoxantrone, and Topoteean, and G₁ phase-dependent agents may include Asparaginase, and Corticosteroids.

Further chemotherapeutic drugs that may be used, in embodiments of the invention are classified by mechanism of action. For example, alkylating agents that impair cell function: nitrogen mustards, are local vesicants, and include mechlorethamine, Mustargen, cyclophosphamide, ifosfamide, Ifex, chlorambucil, and Leukeran; nitrosoureas, which are distinguished by their high lipid solubility and chemical instability, rapidly and spontaneously decompose into two highly reactive intermediates: chloroethyl diazohydroxide and isocyanate; platinum agents include Cisplatin, Platinol, Carboplatin and Paraplatin; and antimetabolites are structural analogs of the naturally occurring metabolites involved in DNA and RNA synthesis that alter the critical pathways of nucleotide synthesis. Natural products possessing antitumor activity that have been isolated from natural substances, such as plants, fungi, and bacteria may also be used in embodiments. For example, antitumor antibiotics, such as Bleomycin or Blenoxane; anthracyclines; epipodophyllotoxins, such as Etoposide, VP-16, VePesid and others, inhibit topoisomerase II activity by stabilizing the DNA-topoisomerase II complex resulting in the inability to synthesize DNA, and the cell cycle is stopped in G₁ phase; vinca alkaloids derived from the periwinkle plant, Vinca rosea; and camptothecin and campothecin analogs which are derived from the Chinese ornamental tree, Camptotheca acuminate and inhibit topoisomerase I interrupting the elongation phase of DNA replication.

The chemotherapeutic drug or agent may also be attached to the susceptor, and such a susceptor would constitute a dual therapy susceptor.

In one embodiment of the invention, thermotherapy may be combined with chemotherapeutic drugs or agents attached to MAB's. Monoclonal antibodies (MAB's) can be bound to a chemotherapy agent. This combination allows for two mechanisms of attacking the cell: 1) the chemical from the chemotherapy, and 2) the immune response from the MAB, Chemotherapy can be more effective when the cells are weakened by the MAB. These agents can be administered prior to, dining, or after thermotherapy administration.

In another embodiment, thermotherapeutic agents may be used in combination with therapies that involve biologic agent such as, for example, antibodies that are not attached to chemotherapeutic agents. For example, an MAB that is not attached to a chemotherapeutic agent may be administered. Such an MAB may induce an immune response against the cancerous tissue which may facilitate treatment.

In still another embodiment, the chemotherapeutic drug or agent is activated during the AMF exposure as it is released from the susceptor due to the inductive heating. The drug or agent can also be destroyed when the AMF is turned on. In an alternative embodiment, the drug or agent is incorporated into a susceptor coating and released when the AMF is applied. Such coating may include one or more layers, where the layers may be of the same or different material, and the drag or agent may be incorporated into one or more of the coating layers.

In yet other embodiments, thermotherapy and chemotherapy may be administered along with an agent that increases the permeability of the blood vessels within the tumor to permit more therapeutic drug to reach and kill substantially more cancer cells. For example, vasopermeation enhancement agents (VEA's) are drugs designed to increase the uptake of cancer therapeutics and imaging agents at the tumor site, potentially resulting in greater efficacy. VEA's work by using monoclonal antibodies, or other biologically active targeting agents, to deliver known vasoactive compounds (i.e., molecules that cause tissues to become more permeable) selectively to solid tumors. Once localized at the tumor site, VEA's alter the physiology and the permeability of the vessels and capillaries that supply the tumor. In pre-clinical studies, drug uptake has been increased up to 400% in solid tumors when VEA's were administered several hours prior to the therapeutic treatment, VEA's are intended for use as a pre-treatment for most existing cancer therapies and imaging agents. VEA's may be effective across multiple tumor types. Examples of VEA's include the commercially available Cotara™ and Oncolym® (Peregrine Pharmaceuticals, Inc., Tustin, Calif.). VEA's can be used with the targeted thermotherapeutic therapy to enhance the blood flow and hence the uptake of susceptors at the tumor cells.

In some embodiment of the invention, thermotherapy may be combined with open or minimally invasive surgery or with other interventional techniques. In such embodiments, the susceptor can be heated with the AMF during the operation or the intervention. The AMF energy source may be a part, of the operational space and thus covered in sterile material. In such instances, all surgical tools are made from non-magnetic materials such as plastic, ceramic, glass or non-magnetic metals or metal-alloys (titan). The AMF energy source may be located next to the sterile surgical site, and the patient can be moved in and out of the AMF energy field, in a manual or automatic manner.

For example, in one embodiment, an organ may be surgically prepared to be lifted to outside the patient's body while it continues to be anatomically and physiologically attached to the body, susceptors may be injected into the organ, and the organ may be irradiated with the AMF extracorporeally. The treated organ is then replaced into the patient's body. Such a technique may allow for enhanced selectivity of the AMF to only the targeted organ, while other parts of the body are unexposed to the AMF. In another embodiment, thermotherapy can be administered at least once prior to, at least partly during, at least once after surgery or other interventional technique, or any combination thereof.

In still other embodiments, thermotherapy may be combined with bone marrow and/or stem cell transplantation. In one embodiment of the invention, thermotherapy is administered prior to, during, or after bone marrow or stem cell transplantation, or any combination thereof. In another embodiment, thermotherapy can be administered to transplanted bone marrow or stem cells excorporeally, prior to transplantation. Bone marrow contains immature cells referred to as stem cells that produce blood cells. Most, stem cells are found in the bone marrow, but some stem cells referred to as peripheral blood stem cells (PBSC's) can be found in the bloodstream. Stem cells can divide to form more stem cells, or they can mature into white blood cells, red blood cells, or platelets. Bone marrow transplantation (BMT) and peripheral blood stem cell transplantation (PBSCT) are procedures that restore stem cells that have been destroyed by high doses of chemotherapy and/or radiation therapy since without healthy bone marrow, the patient is no longer able to make the blood cells needed to carry oxygen, defend against infection, and prevent, bleeding. Stem cells that, have been destroyed by treatment are replaced using BMT and PBSCT.

In yet other embodiments, thermotherapy may be combined with photodynamic therapy (PDT). PDT is based on light-sensitive molecules, photosensitizers (PS's) that concentrate in tumor tissues. When irradiated with light of an appropriate wavelength, PS's absorb light and become excited, transferring their energy to nearby molecular oxygen to form reactive oxygen species (ROS's), which in turn oxidize and damage vital components of nearby tumor cells. In embodiments, susceptors may be administered to a subject prior to, during or following PDT and activated either simultaneously or separately from one another, and in other embodiments, susceptors may be coated with photosensitive drugs. For example, in one embodiment, silica-based or other optically activated nanoparticles with a magnetic core may be produced and a PDT drug may be used to coat these nanoparticles. These susceptors may then be irradiated with light to activate the drug, and they are irradiated later with the AMF of the targeted thermotherapy system to further destroy the target via heat. The susceptors may also be irradiated with light and with AMF simultaneously. In certain embodiments, photodynamic therapy in combination with, thermotherapy may be used alone or in combination with chemotherapy, surgery or both.

The therapies and combined therapies described hereinabove can be further combined in any combination as deemed suitable for the patient. In embodiments of the invention. There may be a disease which can be treated with two (dual therapy) or more therapies. The targeted thermotherapy using nano-sized particles in combination with another therapy may treat two or more diseases.

As noted above, the present invention is applicable to thermotherapeutic compositions for treating disease material, and methods of targeted therapy utilizing such compositions. The present invention should not be considered limited to the particular embodiments described above, but rather should be understood to cover all aspects of the invention as fairly set out in the attached claims. Various modifications, equivalent processes, as well as numerous structures to which the present invention may be applicable, will be readily apparent to those skilled in the art to which the present invention is directed upon review of the present specification. The claims are intended to cover such modifications and devices.

### EXAMPLES

A variety of analytical techniques have been applied to physically characterize the susceptors described herein:

Analytical ultracentrifugation (AUC) was used to determine a density for the nanoparticles and an accurate size and size distribution for the iron oxide core.

Photon Correlation Spectroscopy (PCS) was used to determine an average size and size distribution of the entire core/shell structure.

The hysteresis loops were measured with a MPMS SQUID Magnetometer from Quantum Design. All of the measurements were made at room temperature (298 K) using a Kel-F liquid capsule holder from LakeShore Cryotronics to hold the colloid, and the field range was from ±3.98 MA/m (±50,000 Oe).

Transmission Electron Microscopy (TEM) was performed on a JEOL JEM3010 TEM at 300 keV. The colloids were diluted to 1/100 by volume and then dropped onto a carbon coated TEM grid to dry.

The Small Angle Neutron Scattering (SANS) experiments were conducted at the NG-3 beam line at the NIST Center for Neutron Research (NCNR) using neutrons with a wavelength of 8.4 Å. Data were collected in transmission mode with a two-dimensional detector at three different sample-to-detector distances in order to span the range of scattering vectors Q from 3×10⁻⁵ to 5×10⁻¹ Å⁻¹. These data were corrected for the background from an empty cell and for distortions in the detector.

To probe smaller Q values, Ultra-SANS (USANS) experiments were performed using the BT5 thermal neutron double-crystal instrument at NCNR. The samples were run for 8 hours each at a neutron wavelength of 2.4 Å. A background from an empty beam run was subtracted from all the data, and the subtracted, data processed to an absolute scale by use of the straight through beam intensities. The Q (wave vector component in the horizontal plane) range corresponds to probing length scales from 500 to 20,000 nm. All of the SANS and USANS measurements were made at room temperature and in zero field. The samples in H₂O were held in 1 mm thick quartz cells while the samples in D₂O were held in 4 mm thick quartz cells. A series of concentrations (not shown due to space considerations) were also used in order to help constrain the parameters for the fitting. A core-shell model was used, to fit the data. All SANS and USANS reduction and fits were performed using interactive IGOR procedures [17].

Specific Absorption Rate (SAR) measurements, to determine the heat dose of the nanoparticles, were made in a modified alternating magnetic field (AMF) calorimeter under varying field amplitudes at a frequency of 150 kHz. SAR values were calculated from the rate of temperature rise measured in the water when the particle suspension was heated by the AMF generated in a solenoid coil after correction for the thermal properties of the calorimeter, coil, and water. The values were normalized for iron content.

The in vivo mouse trials were performed in an AMF inductor that confines high-amplitude magnetic fields to a 1 cm wide band of the interior of a 3.5 cm internal diameter induction coil (FIG. 1). Mice were subjected to varying combinations of AMF by adjusting amplitude and duration of exposure. The duty cycle was 100% (always on) and the frequency was fixed at 150 kHz. The duration of exposure was limited to 15 minutes, or when the rectal temperature of the mouse reached 41.5° C. The nanoparticles were directly injected into the central portion of the tumors over a 5 minute period. Temperatures were continuously recorded using 0.4 mm diameter fiberoptic temperature assessment probes which are not RF-sensitive and were placed in the center of the tumor, immediately adjacent to the tumor and in the rectum.

### Example 1

Two different samples of magnetic susceptors having iron oxide (magnetite) cores coated with dextran to form a shell and having a diameter less than 50 nm were synthesized using high-pressure homogenization according to the core/shell method described in U.S. Patent Application No. 2005/0271745. The two samples are nominally identical in their cores. However, the dextran shell layer varies: Sample A includes a single dextran layer while Sample B includes a double dextran layer.

AUC yielded a density of 3.20 g/cm³, which is slightly less than that of bulk iron oxide at 5.18 g/cm³, and a size distribution of 44±13 nm for the nanoparticle cores. PCS yielded a larger size and size distribution of 96.5±32.4 nm. This number is the same whether it is determined by intensity or by volume. However, the PCS instrument estimates a hydrodynamic radius based upon a Stokes-Einstein sphere moving through the solvent and, thus, includes an estimate of the thickness of the dextran layer infiltrated with solvent.

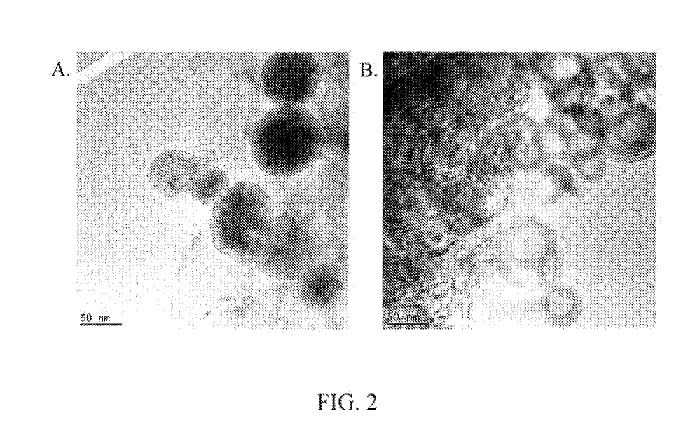

A dextran length of 26 nm is reasonable for the 40,000 Dalton dextran used. The AUC data also agree with the TEM images (FIG. 2) that show a core diameter of ˜50 nm. The dextran layer thickness cannot be determined from the TEM as (i) it is a dried sample and (ii) it is difficult to separate the amorphous dextran from the amorphous carbon film coating the TEM grid at this excitation energy. Close examination of the TEM images reveal the presence of a dark ring at the edge of the iron oxide core in the Sample B (FIG. 1B.) which is not present in the Sample A (FIG. 1A.). Rather, the core of Sample A (FIG. 1A.) appears denser than the edge, as expected for a sphere. This dark ring in Sample B (FIG. 1B.) may be due to one of two things: the nanoparticles are thicker at the edge than in the center or the edge has a different density than the core. Given the density of the iron oxide is only about 62% that of the bulk, the ring is probably due to the edge having a different density than the core.

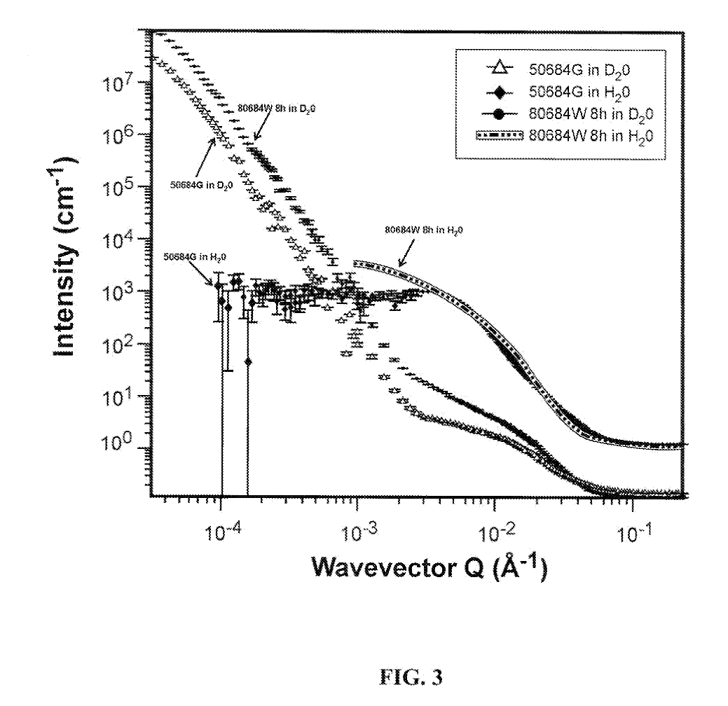

The SANS/USANS data are also in reasonable agreement with the TEM. The data for both Sample A and Sample B under different contrast conditions are shown in FIG. 3. H₂O and D₂O each highlight different features, of the system by varying the sample contrast. In the case of H₂O, the scattering appears to be dominated by the large contrast between iron oxide and H₂O, whereas there is less contrast with dextran. In D₂O, the intensity of scattering from the core is much reduced while the contrast with dextran is enhanced. Both of these samples in D₂O show a strong scattering intensity at low Q that may be due to the: presence of a dextran network acting to bind particles in large scale aggregates. This interpretation agrees with other observations of dextran solutions. However, the D₂O SANS data also highlights the significant differences between the two cores. A polydispersed core-shell model was used to fit the H₂O data by keeping the ratio of core to shell sizes constant. This yields a total particle diameter of 28.30±0.02 nm. This is smaller than the size seen by either PCS or AUC and this difference is attributed to the fact that neutron scattering is sensitive to the first moment of the distribution of radii in a polydispersed system, whereas PCS and AUC are sensitive to the third moment. Furthermore, it is possible that the radial density profile of the particles is not simply a uniform core and shell, as seen from the TEM. In addition, it is expected that there exists a decreasing density gradient of dextran with increasing radius.

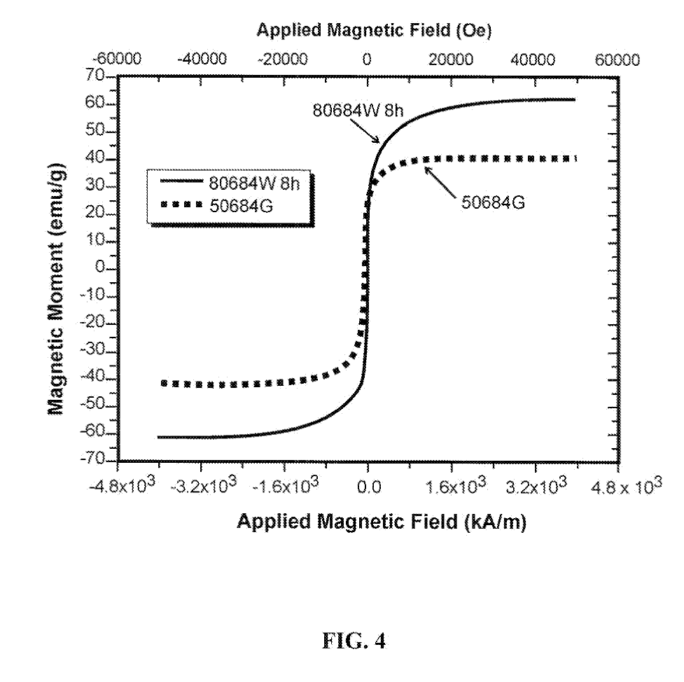

The magnetic properties of the system were characterized by measuring the hysteresis loops at room temperature. These loops (FIG. 4) have been normalized to the mass of particles present in the colloid using the mass of solution added to the liquid capsule holder, its density as determined with an Anton Paar DMA 5000 Densitomete, and mass concentration of material in the colloid as determined by freeze-drying 1 ml of colloid. The most prominent point is that the saturation magnetization of Sample B is 41.08±0.03 kA-m²/g, 33% less than that of the Sample A of 61.64±0.03 kA-m²/g. This significant difference in magnitude may be related to the darker ring seen in the TEM.

The SAR values were measured for H=85.9 kA/m (1080 Oe) and f=150 kHz and are normalized to iron concentration. Sample B had a colloidal concentration of 5 mg/ml while Sample A had a slightly higher concentration of 5.5 mg/ml. Sample B had a measured SAR of 209 W/g of Fe while Sample A had a measured SAR of 537 W/g of Fe, a difference of a factor of 2.5. Most of this difference can be attributed to the difference in the saturation magnetization, although not all. Additional contributions may originate from the collective behavior of the nanoparticles due to differences in their interactions.

In vivo characterization to quantify the efficacy of this treatment in five groups are described in Table 1. The first four groups study the effect of field amplitude, while group 5 is a control group with no iron oxide nanoparticles injected with a field applied. The last three columns also contain the maximum temperature achieved, the normalized rate of heat dosage deposited, and the total normalized heat dosage applied. Conventionally, it is expected that the higher heat dosage should generate the larger temperature change and, therefore, greater efficacy. However, as the mechanism for how nanoparticle generated heat damages tumor cells is unknown and since the dissipation of nanoparticle delivered heat is also not well characterized, this is too simplistic of a viewpoint, instead, it appears from this data that the maximum temperature occurs with the largest dosage rate, which occurs with the largest field amplitude and the shortest on time. This may be a result of the physiological response of the mouse. In any endothermic animal, body temperature is regulated by expanding blood vessels, thermal washout, or by shivering and contraction of blood vessels close to the skin to generate/conserve heat internally. The former process will definitely be a factor in removing convective heat that is generated locally from the iron oxide nanoparticles. Because this is a dynamic process, the faster that heat can be deposited into the local area, the greater the temperature change before the physiological response can remove it. However, other physiological responses, such as damage to the blood vessel from heat at higher temperatures of 46° C.-48° C., may restrict or even stop such blood flow creating a higher than expected heating situation, thereby limiting the applicability of this simple view. For this reason, the physical characterization is insufficient to determine efficacy. Physiological responses must be considered and in vivo studies performed in order to truly determine the efficacy of a treatment.

TABLE 1

| | | | | | Heat Dosage | Maximum |
|---|---|---|---|---|---|---|
| | Amplitude | Time On | Particle Dose | Total Heat Dose | Rate | Temperature |
| Group | (Oe) | (s) | (mg of Fe) | (J/g-tumor) | (J/s/g-tumor) | (° C.) |
| 1 | 400 | 900 ± 0 | 722 ± 50 | 702.50 ± 0.91 | 0.780 | 37.13 ± 1.27 |
| 2 | 550 | 1002 ± 164 | 845 ± 183 | 905.98 ± 137.37 | 0.904 | 45.27 ± 2.93 |
| 3 | 550 | 926 ± 100 | 436 ± 67 | 412.19 ± 43.05 | 0.445 | 46.80 ± 2.48 |
| 4 | 700 | 699 ± 276 | 976 ± 236 | 669.63 ± 256.51 | 0.958 | 51.16 ± 2.40 |
| 5 | 550 | 1200 ± 0 | N/A | N/A | N/A | 40.12 ± 2.31 |

The saturation magnetization, particle structure, and interparticle interactions all affect the SAR. However, each contributes in different ways and with different magnitudes, and may even be in competition with each other. The biological response in our preliminary studies correlates with the measured intratumoral temperature and thermal dose (time and temperature), leading these nanoparticles to appear to have a “global” thermotherapeutic effect, similar to that of conventional hyperthermia. Finally, the physiological effects (e.g., dynamic heat transport mechanisms) which normally have a major influence on the efficacy of conventional hyperthermia treatment may not have the same role in nanoparticle hyperthermia. This is likely due to the fact that the heat source for nanoparticle hyperthermia is internal rather than external for conventional hyperthermia and that the cellular targets for nanoparticle hyperthermia may well be different. Further work is necessary to understand the mechanisms of heat damage and heat dissipation in mammals.

### Example 2

Two different samples of magnetic susceptors having iron oxide (magnetite) cores coated with dextran to form a shell and having a diameter less than 50 nm were synthesized using high-pressure homogenization according to the core/shell method described in U.S. Patent Application No. 2005/0271745. The two samples are nominally identical in their cores with dextran layers that vary: Sample B includes a double dextran layer and Sample C was coated and includes a single dextran layer.

AUC yielded a density of 3.20 g/cm³, which is slightly less than that of bulk iron oxide at 5.18 g/cm³, and a size distribution of 44±13 nm for the nanoparticle core. PCS yielded a larger size and size distribution of 92±14 nm. This number is the same whether it is determined by intensity or by volume. However, the PCS instrument estimates a hydrodynamic radius based upon a Stokes-Einstein sphere moving through the solvent and, thus, includes an estimate of the thickness of the dextran layer infiltrated with solvent. A dextran length of 24 nm is reasonable for the 40,000 Dalton dextran used. The AUC data also agree with the TEM images showing a core diameter of ˜50 nm. As described above, the dextran layer thickness cannot be determined from the TEM.

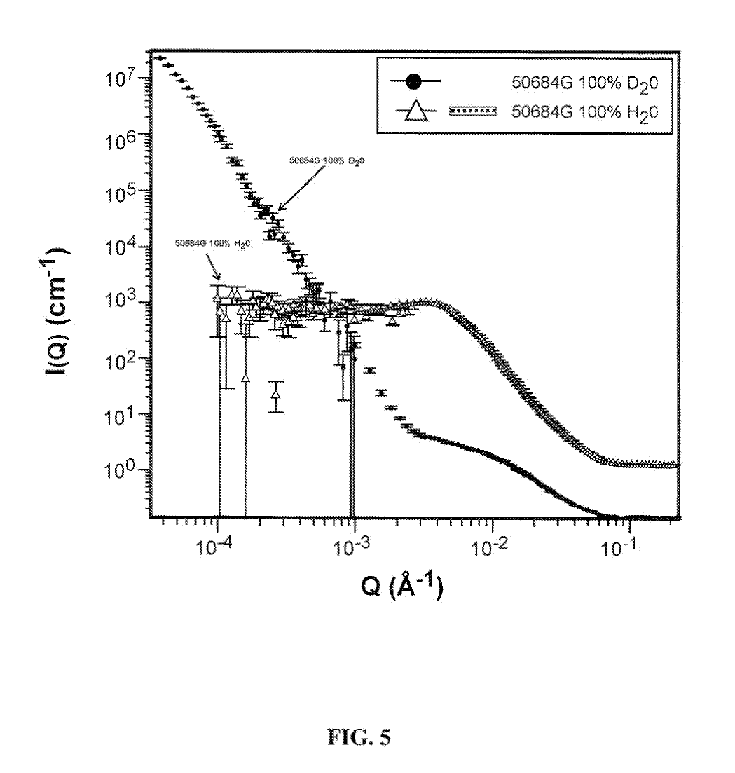

The SANS/USANS data are also in reasonable agreement with these numbers. Sample B data, both H₂O and D₂O, are shown in FIG. 5. H₂O and D₂O each highlight different features of the system by varying the sample contrast. In the case of H₂O, the scattering is dominated by the large contrast between iron oxide and H₂O, whereas there is less contrast with dextran. In D₂O, the intensity of scattering from the core is much reduced while the contrast with dextran is enhanced. Sample B in D₂O data show a strong scattering intensity at low Q that may be due to the presence of a dextran network acting to bind particles in large scale aggregates. This interpretation agrees with other observations of dextran solutions. A polydispersed core-shell model was used to fit the H₂O data by keeping the ratio of core to shell sizes constant. This yields a total particle diameter of 28.30±0.02 nm. This is smaller than the size seen by either PCS or AUC and this difference is attributed to the fact that neutron scattering is sensitive to the first moment of the distribution of radii in a polydispersed system, whereas PCS and AUC are sensitive to the third moment. Furthermore, it is possible that the radial density profile of the particles may not be a uniform core and shell. For instance, there may be a decreasing density gradient of dextran with increasing radius. Finally, the hard sphere interaction radius is determined to be 69.5±0.2 nm indicating that there is an interaction on a length scale longer than the particle size visible to neutrons.

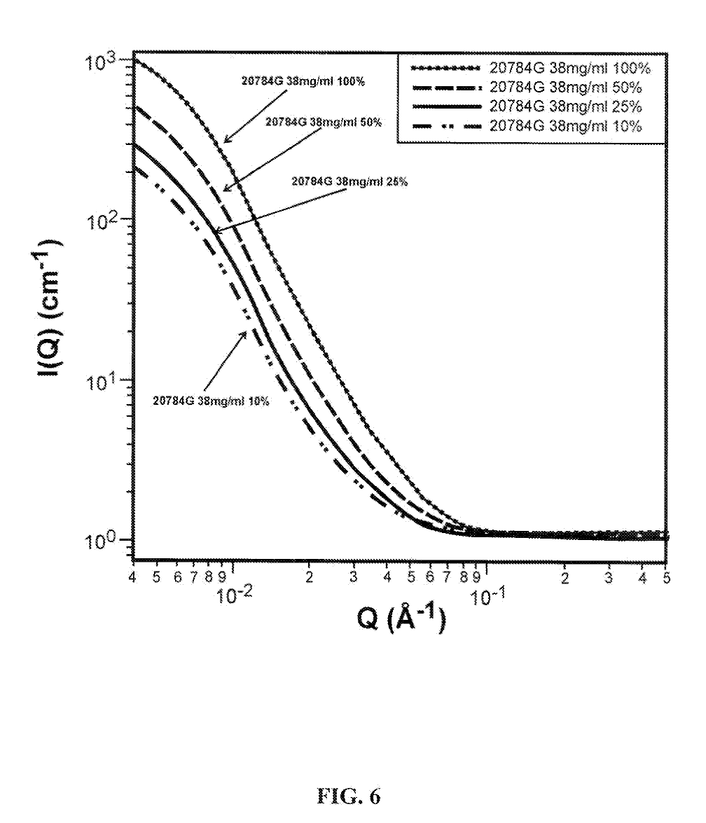

The SANS data from Sample C are shown in FIG. 6. The concentration series in H₂O is shown, while the D₂O data that are similar to those in FIG. 5 are excluded. Here the diameter is 27.2±0.5 nm, similar to that found for Sample B, while the interaction radius is >200 nm, a factor of 3 larger than for Sample B. This increase in the interaction radius does not appear to be due to a change in average diameter as determined earlier by both AUC and PCS, nor by a change in volume fraction. From SANS/USANS, the volume fractions of both samples are nearly equal (0.1075±0.0005 for Sample B and 0.1050±0.0003 for Sample C), as is the polydispersity (0.6), which is an indicator of the size distribution. This difference in interaction radius with no difference in particle dimensions indicates that a simple hard sphere interaction model is not physically correct and a better model would take into account steric, electrostatic, and magnetic interactions.

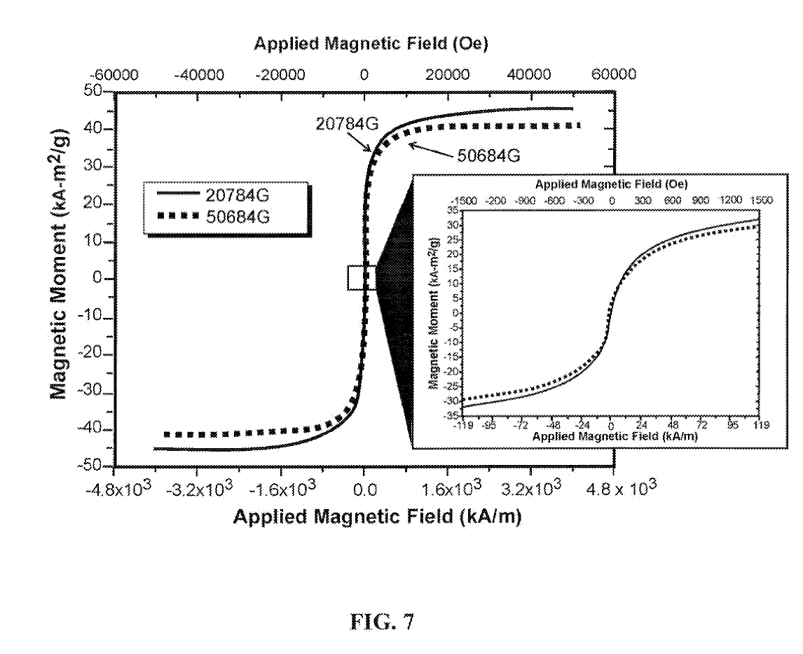

The magnetic properties of the system were characterized by measuring the hysteresis loop at room temperature. These loops (FIG. 7) have been normalized to the mass of particles present in the colloid using the mass of solution added to the liquid capsule holder, its density as determined with an Anton Paar DMA 5000 Densitometer, and mass concentration of material in the colloid as determined by freeze-drying 1 ml of colloid. The most prominent point is that the saturation magnetization of Sample B is 4108±0.03 kA-m²/g, slightly less than that of Sample C (45.34±0.02 kA-m²/g). Other than this slight difference in magnitude, the shapes of the two hysteresis loops are nearly identical. It is noteworthy that the SANS data exhibit significantly different interaction radii in zero field for the two samples, although the SQUID data show nearly identical magnetic moments at the fields used in the SAR measurements.

The SAR values were measured for H=86 kA/m (1.080 Oe) and f=150 kHz using colloids of nominally equal concentrations which are then normalized to iron concentration. Here we see the most striking difference. Sample B has a measured SAR of 1075 W/g of Fe while Sample C has a measured SAR of 150 W/g of Fe, a difference of a factor of 7. This cannot be attributed to a difference in the saturation magnetizations as these are shown to have the opposite trend and to differ only by 10%, nor can this be attributed to differences in the physical size or size distribution as these have been shown through three physical techniques (TEM, AUC, and PCS.) to be nearly the same. Therefore, the primary difference appears to be in the SANS/USANS data in the interaction behavior. In particular, the double dextran layer of Sample B appears to have a much smaller interaction radius, by nearly a factor of 3. This smaller interaction radius may have a two-fold effect: (1) the dipolar interactions would be significantly stronger enabling the nanoparticles to couple their behavior under an oscillating field, thereby amplifying the heating, and (2) the smaller interaction radius would mean that more particles are grouped closer together, enhancing the local heat output in a smaller area.

Although the magnetic properties of a sample (saturation magnetization, anisotropy, and volume) are expected to have significant effects on the heat dose supplied by magnetic nanoparticles under the influence of an alternating magnetic field, the individual nanoparticle properties are not the only consideration. The behavior of the collection of magnetic nanoparticles is equally critical in determining the heat dose, as demonstrated by two nominally identical samples of ˜50 nm iron oxide core/shell nanoparticles. The tightly associated system has a measured SAR of 1075 W/g of Fe while the more loosely associated system has an SAR of 150 W/g of Fe.

Although the present invention has been described in considerable detail with reference to certain preferred embodiments thereof other versions are possible. Therefore the spirit and scope of the appended claims should not be limited to the description and the preferred versions contained within this specification.

What is claimed is:

1. A therapeutic composition comprising:
a plurality of untargeted magnetic nanoparticles having an interaction radius of from about 100 nm to about 50 nm; and
a pharmaceutically acceptable carrier.

2. The therapeutic composition of claim 1, wherein the plurality of untargeted magnetic nanoparticles comprises stable single-magnetic domain nanoparticles, superparamagnetic particles and combinations thereof.

3. The therapeutic composition of claim 1, wherein the plurality of untargeted magnetic nanoparticles are apparently thermally blocked when exposed to a magnetic field and become heated.

4. The therapeutic composition of claim 1, wherein the plurality of untargeted magnetic nanoparticles have an average particle size of less than about 1 μm.

5. The therapeutic composition of claim 1, wherein the plurality of untargeted magnetic nanoparticles have an average particle size of from about 0.1 nm to about 800 nm.

6. The therapeutic composition of claim 1, wherein the plurality of untargeted magnetic nanoparticles have a polydispersity of from about 0.1 to about 1.5.

7. The therapeutic composition of claim 1, wherein the plurality of untargeted magnetic nanoparticles are prepared from a material comprising Fe₃O₄, γ-Fe₂O₃, FeCo/SiO₂, Co₃₆C₆₄, Bi₃Fe₅O₁₂, BaFe₁₂O₁₉, NiFe, CoNiFe, Co—Fe₃O₄, FePt—Ag and combinations thereof.

8. The therapeutic composition of claim 1, wherein the plurality of untargeted magnetic nanoparticles comprise a core and a coating.

9. The therapeutic composition of claim 8, wherein the core comprises a material selected from Fe₃O₄, γ-Fe₂O₃, FeCo/SiO₂, Co₃₆C₆₄, Bi₃Fe₅O₁₂, BaFe₁₂O₁₉, NiFe, CoNiFe, Co—Fe₃O₄, FePt—Ag and combinations thereof.

10. The therapeutic composition of claim 8, wherein the coating comprises a material selected from polymers, biological materials, inorganic coating materials and combinations thereof.

11. The therapeutic composition of claim 10, wherein the polymers are selected from acrylates, siloxanes, styrenes, acetates, akylene glycols, alkylenes, alkylene oxides, parylenes, lactic acid, glycolic acid, hydrogel polymer, histidine-containing polymer, and combinations thereof.

12. The therapeutic composition of claim 10, wherein the biological materials are selected from heparin, heparin sulfate, chondroitin sulfate, chitin, chitosan, cellulose, dextran, alginate, starch, carbohydrate, glycosaminoglycan, extracellular matrix proteins, proteoglycans, glycoproteins, albumin, gelatin and combinations thereof.

13. The therapeutic composition of claim 10, wherein the inorganic coating materials are selected from metals, metal alloys and ceramics.

14. The therapeutic composition of claim 8, wherein the core is magnetite and the coating is dextran.

15. The therapeutic composition of claim 14, wherein the coating comprises at least two layers of dextran.

16. The therapeutic composition of claim 1, wherein the plurality of untargeted magnetic nanoparticles have an interaction radius of from about 200 nm to about 25 μm.

17. The therapeutic composition of claim 1, wherein the plurality of untargeted magnetic nanoparticles have a saturation magnetism of from about 10 kA-m²/g to about 100 kA-m²/g.

18. The therapeutic composition of claim 1, wherein the plurality of untargeted magnetic nanoparticles have a specific absorption rate (SAR) of from about 100 W/g to about 1500 W/g when exposed to an alternating magnetic field.

19. The therapeutic composition of claim 1, wherein the pharmaceutically acceptable carrier is selected from water, buffered water, saline, Ringers solution, glycine, hyaluronic acid, dextrose, albumin solution, oils or combinations thereof.

20. The therapeutic composition of claim 19, further comprising one or more additives selected from stabilizers, antioxidants, osmolality adjusting agents, buffers, pH adjusting agents, chelants, calcium chelate complexes, salts or combinations thereof.

21. The therapeutic composition of claim 1, wherein the therapeutic composition is formulated as a liquid, a gel, an ointment, a lotion, a solid, or a semi-solid.

22. The therapeutic composition of claim 1, further comprising targeted magnetic nanoparticles.

23. The therapeutic composition of claim 1, further comprising one or more secondary agents selected from chemotherapeutic agents, radiation therapy agents, vasopermeation enhancement agents, anti-inflammatory agents, anesthetics, analgesics, sedatives, antibiotics and combinations thereof.

24. A method for treating tumorigenic tissue comprising:
administering to a patient in need of treatment an effective amount of a therapeutic composition comprising:
a plurality of untargeted magnetic nanoparticles having an interaction radius of from about 100 nm to about 50 μm; and
a pharmaceutically acceptable carrier or excipient; and
exposing the patient to an energy capable of inducing heating of the plurality of untargeted magnetic nanoparticles.

25. The method of claim 24, wherein the tumorigenic tissue is a solid tumor.

26. The method of claim 24, wherein administering comprises contacting the tumorigenic tissue with the therapeutic composition directly.

27. The method of claim 24, wherein administering comprises applying the therapeutic composition directly to the tumorigenic tissue.

28. The method of claim 24, wherein administering comprises injecting a tumor with the therapeutic composition.

29. The method of claim 24, wherein the method is carried out in combination with radiation therapy, chemotherapy, external beam therapy, surgery, photodymanic therapy (PDT), therapy using biological agents or a combination thereof.

30. The method of claim 24, wherein the plurality of untargeted magnetic nanoparticles have an interaction radius of from about 200 nm to about 25 μm.

31. The method of claim 24, wherein the energy is selected from alternating magnetic field (AMF), microwave energy, acoustic energy and combinations thereof.

32. The method of claim 24, wherein the energy is an alternating magnetic field (AMF).

33. The method of claim 32, wherein the alternating magnetic field has a frequency range of from about 80 kHz to about 800 kHz.

34. The method of claim 32, wherein the alternating magnetic field has an amplitude of from about 1 kA/m to about 120 kA/m.

35. A method for treating joint inflammation comprising:
administering to a patient in need of treatment an effective amount of a therapeutic composition comprising:
a plurality of untargeted-magnetic nanoparticles having an interaction radius of from about 100 nm to about 50 μm; and
a pharmaceutically acceptable carrier or excipient; and
exposing the patient to an energy capable of inducing heating of the plurality of untargeted magnetic nanoparticles.

36. The method of claim 35, wherein administering comprises contacting inflamed synovial tissue, scar tissue, immune cells and combinations thereof with the therapeutic composition directly.

37. The method of claim 35, wherein administering comprises applying the therapeutic composition directly to the joint.

38. The method of claim 35, wherein administering comprises injecting the joint with the therapeutic composition.

39. The method of claim 35, wherein administering further comprises administering one or more of an anti-inflammatory agent, anesthetic, analgesic, sedative, antibiotic, or combination thereof.

40. The method of claim 35, wherein the plurality of untargeted magnetic nanoparticles have an interaction radius of from about 200 nm to about 25 μm.

41. The method of claim 35, wherein the energy is selected from alternating magnetic field (AMF), microwave energy, acoustic energy and combinations thereof.

42. The method of claim 35, wherein exposing comprises applying an alternating magnetic field (AMF) to at least a portion of the patient.

43. The method of claim 42, wherein the alternating magnetic field has a frequency range of from about 80 kHz to about 800 kHz.

44. The method of claim 42, wherein the alternating magnetic field has an amplitude of from about 1 kA/m to about 120 kA/m.

45. The method of claim 35, wherein the joint inflammation is caused as a result of injury, disease, arthritis and combinations thereof.

46. The method of claim 45, wherein the arthritis is selected from general arthritis, rheumatoid arthritis, osteoarthritis, tendonitis, bursitis, fibromyalgia and combinations thereof.

47. The method of claim 45, wherein the disease is selected from gout, lupus, rickets, ankylosing spondylitis, Sjogrens syndrome and combinations thereof.
